(19) United States
(12) Patent Application Publication
(10) Pub. No.: US 2006/0263770 A1
(43) Pub. Date: Nov. 23, 2006

(54) Novel receptor trem (triggering receptor expressed on myeloid cells) and uses thereof

(76) Inventors: Marco Colonna, St. Louis, MO (US); Paola Panina, Milano (IT)

Correspondence Address: FOLEY AND LARDNER LLP;SUITE 500, 3000 K STREET NW, WASHINGTON, DC, 20007, US

(21) Appl. No.: 10/508,663

(22) PCT Filed: Mar. 21, 2003
(86) PCT No.: PCT/GB03/01231

§ 371 Date: Feb. 2, 2005

Related U.S. Application Data
(60) Provisional application No. 60/366,525, filed on Mar. 22, 2002.

(51) Int. Cl. C12Q 1/68; G01N 33/567; C07H 21/04; C12P 21/06; C07K 14/705; C07K 16/28

(52) U.S. Cl. 435/6; 435/7.2; 435/69.1; 435/320.1; 435/252.3; 435/325; 530/350; 530/388.22; 536/23.5

(57) ABSTRACT

Novel activating receptors of the Ig super-family expressed on human myeloid cells, called TREM(s) (triggering receptor expressed on myeloid cells) are provided. Specifically, two (2) members of TREMs, TREM-4 (alpha and beta) and TREM-5 are disclosed. TREM-4 is a transmembrane glycoprotein expressed selectively in the endothelium of capillaries, in the heart and in the testis. Use of TREM-4 in treatment and diagnosis of various inflammatory diseases and heart diseases and male infertility are also provided. TREM-5 is also a transmembrane glycoprotein expressed selectively in bone marrow-derived population of leukocytes, in particular dendritic cells, and may be upregulated in certain conditions, such as cell activation, inflammation or aberrant dendritic cell function. Blockade of TREM-5 with monoclonal antibodies or soluble TREM-5-HuIgG fusion protein may reduce or block skin diseases or dendritic cell associated disorders.

### 1. INTRODUCTION

This invention relates generally to new activating receptors of the Ig super-family expressed on human myeloid cells, called TREM (triggering receptor expressed on myeloid cells) which are involved in inflammatory responses. Specifically, this invention relates to two (2) members of the TREM family, TREM-4 (including TREM-4-alpha and TREM-4-beta) and TREM-5.

### 2. BACKGROUND OF THE INVENTION

Inflammatory responses to bacterial and fungal infections are primarily mediated by neutrophils and monocytes (Medzhitov, R. & Janeway, C., Jr., 2000, Innate immunity. N. Engl. J. Med. 343:338-44; Hoffmann, J. A., Kafatos, F. C., Janeway, C. A. & Ezekowitz, R. A., 1999, Phylogenetic perspectives in innate immunity. Science 284:1313-8). These cells express pattern recognition receptors (PRR) which recognize conserved molecular structures shared by groups of microorganisms (Aderem, A. & Ulevitch, R. J., 2000, Toll-like receptors in the induction of the innate immune response. Nature 406:782-7; Beutler, B., 2000, Endotoxin, toll-like receptor 4, and the afferent limb of innate immunity. Curr. Opin. Microbiol. 3:23-8). Engagement of PRRs by microbial products activate signaling pathways which control the expression of a variety of genes. These inducible genes encode proinflammatory chemokines and cytokines and their receptors, as well as adhesion molecules and enzymes that produce low molecular weight proinflammatory mediators and reactive oxygen species. The combined action of all these products presumably leads to elimination of the infectious agents and tissue repair. However, excessive secretion of pro-inflammatory mediators, together with overexpression of their receptors, cause excessive autocrine/paracrine activation of neutrophils and monocytes, leading to tissue damage and septic shock (Bone, R. C., 1991, The pathogenesis of sepsis. Ann. Intem. Med. 115:457-69; Beutler, B., Milsark, I. W. & Cerami, A. C., 1985, Passive immunization against cachectin/tumor necrosis factor protects mice from lethal effect of endotoxin. Science 229:869-71; Morrison, D. C. & Ryan, J. L., 1987, Endotoxins and disease mechanisms. Annu. Rev. Med. 38:417-32; Tracey, K. J. et al., 1986, Shock and tissue injury induced by recombinant human cachectin. Science 234:470-4; Glauser, M. P., Zanetti, G., Baumgartner, J. D. & Cohen, J., 1991, Septic shock: pathogenesis. Lancet 338:732-6). Thus, the regulation of neutrophil and monocyte activation by stimulatory receptors and their ligands is crucial to the outcome of host inflammatory responses to infections.

Neutrophil- and monocyte/macrophage-mediated inflammatory responses can be stimulated through many receptors with different structures and specificities (Rosenberg, H. F., and J. I. Gallin, 1999, Inflammation. In Fundamental Immunology, 4^{th} Ed. W. E. Paul, ed. Lippincott-Raven, Philadelphia p. 1051). These include G protein-linked seven-transmembrane domain receptors specific for either fMLP, lipid mediators, complement factors, or chemokines, the Fc and complement receptors, the CD14 and Toll-like receptors for LPS, as well as the cytokine receptors for IFN-γ and TNF-α (Ulevitch, R. J., and P. S. Tobias, 1999, Recognition of Gram-negative bacteria and endotoxin by the innate immune system. Curr. Opin. Immunol. 11:19). In addition, engagement of these receptors can up-regulate or “prime” the responsiveness of myeloid cells to other stimuli, potentiating the inflammatory response (Downey, G. P., T. Fukushima, L. Fialkow, and T. K. Waddell, 1995, Intracellular signaling in neutrophil priming and activation. Semin. Cell Biol. 6:345).

Neutrophils and macrophages express additional activating receptors, but their role in inflammation is unknown. These receptors belong either to the Ig superfamily (Ig-SF), such as Ig-like transcripts (ILT)/Ieukocyte Ig-like receptors (LIR)/monocyte/macrophage Ig-like receptors (MIRs), paired Ig-like receptor (PIR-As), and signal regulatory protein β1 (SIRPβ1), or to the C-type lectin superfamily, such as myeloid DAP12-associating lectin-1 (MDL-1) (Nakajima, H., J. Samaridis, L. Angman, and M. Colonna, 1999, Human myeloid cells express an activating ILT receptor (ILT1) that associates with Fc receptor γ-chain. J. Immunol. 162:5; Yamashita, Y., M. Ono, and T. Takai, 1998, Inhibitory and stimulatory functions of paired Ig-like receptor (PIR) family in RBL-2H3 cells. J. Immunol. 161:4042; Kubagawa, H., C. C. Chen, L. H. Ho, T. S. Shimada, L. Gartland, C. Mashburn, T. Uehara, J. V. Ravetch, and M. D. Cooper, 1999, Biochemical nature and cellular distribution of the paired immunoglobulin-like receptors, PIR-A and PIR-B. J. Exp. Med. 189:309; Dietrich, J., M. Cella, M. Seiffert, H.-J. Buhring, and M. Colonna, 2000, Signal-regulatory protein β1 is a DAP12-associated activating receptor expressed in myeloid cells. J. Immunol. 164:9; Bakker, A. B., E. Baker, G. R. Sutherland, J. H. Phillips, and L. L. Lanier, 1999, Myeloid DAP12-associating lectin (MDL)-1 is a cell surface receptor involved in the activation of myeloid cells. Proc. Natl. Acad. Sci. USA 96:9792). Typically, all of these receptors bear some homology with activating NK cell receptors (Lanier, L. L., 1998, NK cell receptors. Annu. Rev. Immunol. 16:359). In particular, they contain a short intracellular domain that lacks docking motifs for signaling mediators and a transmembrane domain with a positively charged amino acid residue. This residue allows pairing with transmembrane adapter proteins, which contain a negatively charged amino acid in the transmembrane domain and a cytoplasmic immunoreceptor tyrosine-based activation motif (ITAM). Specifically, ILT/LIR/MIR and PIRs are coupled with the γ-chain of the Fc receptors (FcRγ) (Nakajima, H., supra; Yamashita, Y., supra; Kubagawa, H., supra), whereas SIRPβ1 and MDL-1 pair with DAP12 (Dietrich, J., supra; Bakker, A. B., supra). Upon ITAM phosphorylation, these adapters recruit protein tyrosine kinases, which initiate a cascade of phosphorylation events that ultimately lead to cell activation.

DAP12-deficient mice exhibit a dramatic accumulation of dendritic cells (DCs) in muco-cutaneous epithelia, associated with an impaired hapten-specific contact sensitivity (Bakker, A. B., Hoek, R. M., Cerwenka A., Blom, B., Lucian, L., McNeil, T., Murray, R., Phillips, L. H., Sedgwick, J. D., and Lanier L. L., 2000, DAP12-deficient mice fail to develop autoimmunity due to impaired antigen priming. Immunity 13:345-53; Tomasello, E., Desmoulins, P. O., Chemin, K., Guia, S., Cremer, H., Ortaldo, J., Love, P., Kaiserlian, D., and Vivier, E., 2000, Combined natural killer cell and dendritic cell functional deficiency in KASRAP/DAP12 loss-of-function mutant mice. Immunity 13:355-64). Furthermore, recent evidence suggests that the interaction between CCR7 (CC family chemokine receptor no. 7) and ELC (Epstein-Barr virus-induced molecule 1 ligand chemokine) triggers DC trafficking to the lymph nodes. In particular, skin DCs from CCR7 −/− mice, as well as in DAP12 −/− mice, are severely impaired in migrating to the draining LNs following activation (Foster, R., Schubel, A., Breiffeld, D., Kremmer, E., Renner-Muller, I., Wolf, E., and Lipp, M., 1999, CCR7 coordinates the primary immune response by establishing functional microenvironments in secondary lymphoid organs. Cell 99:23-33). However, the DAP12-associated receptor responsible for these phenotypes is yet unknown.

The recent discovery of a new DAP12-associated receptor on NK cells, called NKp44 (Cantoni, C., C. Bottino, M. Vitale, A. Pessino, R. Augugliaro, A. Malaspina, S. Parolini, L. Moretta, A. Moretta, and R. Biassoni, 1999, NKp44, a triggering receptor involved in tumor cell lysis by activated human natural killer cells, is a novel member of the immunoglobulin superfamily. J. Exp. Med. 189:787), suggested the possible existence of yet unknown DAP12-associated receptors also on other cells involved in innate responses.

The present inventors have identified new immunoglobulin-super-family (Ig-SF) receptors designated as TREMs (triggering receptor expressed on myeloid cells), that are involved in the regulation of a variety of cellular responses, especially inflammatory responses as well as trafficking of DCs.

### 3. SUMMARY OF INVENTION

The present invention is based upon the inventors' identification of two cDNA molecules which encode triggering receptors expressed on myeloid cells (TREM-4, including TREM-4-alpha: SEQ ID NO:1 and TREM-4-beta: SEQ ID NO:2 which represent alternatively spliced forms of the same transcript; and TREM-5: SEQ ID NO:3). These molecules are expressed on human myeloid cells and are novel transmembrane proteins of the immunoglobulin superfamily (Ig-SF).

TREM-4-alpha and TREM-4-beta are transmembrane glycoproteins having the amino acid sequence of SEQ ID NO:4 and SEQ ID NO:5, respectively, which are selectively expressed in the endothelium of capillaries and in the testis. The experiments and results presented herein utilize reagents that cross-react with nucleic acid and protein sequences of both variants of TREM-4. Thus the term TREM-4 is used herein to indicate both gene products. TREM-4 is strongly expressed in the capillaries of subcutaneous adipose tissue, lymph nodes, thymus, as well as in liver sinusoid endothelium cells, but not in the capillaries of dermis, lung and placenta, the endothelium of arteries, arterioles, veins, venules and lymphatic vessels. TREM-4 may be differentially expressed in different types of testicular germ cell tumors (seminomas and nonseminomatous germ cell tumors, e.g., NSGCTs). TREM-4 has utility in the regulation of inflammation, neoplastic transformation, myeloid cells, atherosclerosis, tumorigenesis, spermatogenesis, and microcirculation.

TREM-5 is a transmembrane glycoprotein having the amino acid sequence of SEQ ID NO:6 which is selectively expressed in bone marrow-derived population of leukocytes and on dendritic cells (DCs). Thus, TREM-5 has utility in the regulation of immune response and dendritic cell function. TREM-5 may be modulated in certain conditions, such as cell activation or inflammation or diseases or disorders associated with aberrant or impaired dendritic cell function, for example autoimmune diseases, allergies, cancer, transplant rejection, immunodeficienies, lympho-proliferative disorders and infectious diseases.

TREM-5 contains a charged lysine residue in the transmembrane region which is reminiscent of that of activating NK cell and myeloid cell receptors that pair with the transmembrane adapter proteins DAP12. Through association with DAP12, TREM-5 may be involved in the pathogenesis of skin diseases such as atopic dermatitis, familial psoriasis, and epidermodysplasia verruciformis. TREM-5 has utility in the regulation of dermal responses to pathogens.

Thus, the members of the TREM family (TREM or TREMs) can be useful in regulating a variety of cellular processes, especially inflammatory responses and cell activation, therefore have a great potential for therapeutic as well as diagnostic uses. The present invention has identified two other TREMs, TREM-1 and TREM-2, which are disclosed in U.S. provisional application Ser. No. 60/277,238, filed Mar. 20, 2001, and U.S. non-provisional application Ser. No. 10/103,423, filed Mar. 20, 2002, both of which are incorporated by reference herein in their entirety.

Accordingly, this invention provides isolated or recombinantly prepared TREMs, or fragments, homologues, derivatives, or variants thereof, as defined herein, which are herein collectively referred to as “peptides of the invention” or “proteins of the invention.” Furthermore, this invention provides nucleic acid molecules encoding the polypeptide of the invention, which are herein collectively referred to as “nucleic acids of the invention” and include cDNA, genomic DNA, and RNA.

Accordingly, this invention provides isolated nucleic acid molecules which comprise or consist of a nucleotide sequence that is about 30%, 35%, 40%, 45%, 50%, 55%, 60%, 65%, 70%, 75%, 80%, 85%, 90%, 95%, or 98% identical to the nucleotide sequence of SEQ ID NO:1, 2, 3, or a complement thereof. In specific embodiments, such nucleic acid molecules exclude nucleotide sequences of accession nos. D788I2, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

This invention further provides isolated nucleic acid molecules which comprise or consist of about 25, 30, 35, 40, 45, 100, 150, 200, 250, 300, 350, 400, 450, 500, 550, 600, 650, 700, 750, 800, 850, 900, 950 or more contiguous nucleotides of the nucleotide sequence of SEQ ID NO:1, or a complement thereof. In specific embodiments, such nucleic acid molecules exclude nucleotide sequences of accession nos. D78812, AI337247, AW139572, AW274906, AW139573, AI394041, AI621023, AI86456, AI968134, AI394092, AI681036, AI962750, AA494171, AA099288, AW139363, AW135801, AA101983, AF196329, AF213457, AF241220, NM006678.1, NM004821.1, and N41388.

This invention further provides isolated nucleic acid molecules which comprise or consist of about 25, 30, 35, 40, 45, 100, 150, 200, 250, 300, 350, 400, 450, 500, 550, 600, 650, 700, 750, 800, 850, 900, or more contiguous nucleotides of the nucleotide sequence of SEQ ID NO:2, or a complement thereof. In specific embodiments, such nucleic acid molecules exclude nucleotide sequences of accession nos. D78812, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

This invention further provides isolated nucleic acid molecules which comprise or consist of about 25, 30, 35, 40, 45, 100, 150, 200, 250, 300, 350, 400, 450, 500, 550, 600, 650 or more contiguous nucleotides of the nucleotide sequence of SEQ ID NO:1, or a complement thereof. In specific embodiments, such nucleic acid molecules exclude nucleotide sequences of accession nos. D78812, AI337247, AW139572, AW274906, AW139573, AI1394041, AI621023, AI186456, AI968134, AI394092, AI681036, AI962750, M494171, AA099288, AW139363, AW135801, AA101983, AF196329, AF213457, AF241220, NM006678.1, NM004821.1, and N41388.

The invention provides isolated polypeptides or proteins which are encoded by a nucleic acid molecule consisting of or comprising a nucleotide sequence that is at least about 30%, 35%, 40%, 45%, 50%, 55%, 60%, 65%, 70%, 75%, 80%, 85%, 90%, 95%, or 98% identical to the nucleotide sequence of SEQ ID NO:1 or a complement thereof, or SEQ ID NO:2 or a complement thereof, or SEQ ID NO:3 or a complement thereof. In specific embodiments, such nucleic acid molecules exclude nucleotide sequences of accession nos. D78812, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

The invention provides isolated polypeptides or proteins which are encoded by a nucleic acid molecule consisting of or comprising a nucleotide sequence that is at least about 25, 30, 35, 40, 45, 100, 150, 200, 250, 300, 350, 400, 450, 500, 550, 600, 650, 700, 750, 800, 850, 900, 950 or more contiguous nucleotides of the nucleotide sequence of SEQ ID NO:1 or a complement thereof. In specific embodiments, such nucleic acid molecules exclude nucleotide sequences of accession nos. D78812, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

The invention provides isolated polypeptides or proteins which are encoded by a nucleic acid molecule comprising a nucleotide sequence that is at least about 25, 30, 35, 40, 45, 100, 150, 200, 250, 300, 350, 400, 450, 500, 550, 600, 650, 700, 750, 800, 850, 900 or more contiguous nucleotides of the nucleotide sequence of SEQ ID NO:2 or a complement thereof. In specific embodiments, such nucleic acid molecules exclude nucleotide sequences of accession nos. D78812, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

The invention provides isolated nucleic acid molecules comprising a nucleotide sequence encoding a protein having an amino acid sequence that is at least about 40%, 45%, 50%, 55%, 60%, 65%, 70%, 75%, 80%, 85%, 90%, 95%, or 98% identical to the amino acid sequence of SEQ ID NO:4, 5 or 6, or fragments, homologues, derivatives, or variants of said protein, or complement of said nucleic acid molecules. In specific embodiments, such nucleic acid molecules exclude nucleotide sequences of accession nos. D788I2, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

The invention provides isolated nucleic acid molecules comprising a nucleotide sequence encoding a protein having an amino acid sequence that comprises or consists of at least about 10, 15, 20, 25, 30, 50, 75, 100, 125, 150, 175, 200, 225, 230 or more contiguous amino acids of SEQ ID NO:4, or fragments, homologues, derivatives, or variants of said protein, or complements of said nucleic acid molecules. In specific embodiments, such nucleic acid molecules exclude nucleotide sequences of accession nos. D788I2, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

The invention provides isolated nucleic acid molecules comprising a nucleotide sequence encoding a protein having an amino acid sequence that comprises or consists of at least about 10, 15, 20, 25, 30, 50, 75, 100, 125, 150, 175, 200, 220 or more contiguous amino acids of SEQ ID NO:5, or fragments, homologues, derivatives, or variants of said protein, or complements of said nucleic acid molecules. In specific embodiments, such nucleic acid molecules exclude nucleotide sequences of accession nos. D788I2, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

The invention provides isolated nucleic acid molecules comprising a nucleotide sequence encoding a protein having an amino acid sequence that comprises or consists of at least about 10, 15, 20, 25, 30, 50, 75, 100, 125, 150, 175, 200 or more contiguous amino acids of SEQ ID NO:6, or fragments, homologues, derivatives, or variants of said protein, or complements of said nucleic acid molecules. In specific embodiments, such nucleic acid molecules exclude nucleotide sequences of accession nos. D78812, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

Furthermore, the invention provides isolated polypeptides or proteins comprising an amino acid sequence that is at least about 40%, 45%, 50%, 55%, 60%, 65%, 70%, 75%, 80%, 85%, 90%, 95%, or 98% identical to the amino acid sequence of SEQ ID NO:4, 5 or 6, or fragments, homologues, derivatives, or variants thereof. In specific embodiments, such polypeptides or proteins exclude polypeptides or proteins encoded by nucleotide sequences of accession nos. D78812, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

The invention provides isolated polypeptides or proteins comprising an amino acid sequence that comprises or consists of at least about 10, 15, 20, 25, 30, 50, 75, 100, 125, 150, 175, 200, 225, 230 or more contiguous amino acids of SEQ ID NO:4, or fragments, homologues, derivatives, or variants thereof. In specific embodiments, such polypeptides or proteins exclude polypeptides or proteins encoded by nucleotide sequences of accession nos. D78812, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

The invention provides isolated polypeptides or proteins comprising an amino acid sequence that comprises or consists of at least about 10, 15, 20, 25, 30, 50, 75, 100, 125, 150, 175, 200, 220, or more contiguous amino acids of SEQ ID NO:5, or fragments, homologues, derivatives, or variants thereof. In specific embodiments, such polypeptides or proteins exclude polypeptides or proteins encoded by nucleotide sequences of accession nos. D78812, AI337247, AW139572, AW274906, AW139573, AI394041, AI621023, AI186456, AI968134, AI394092, AI681036, AI962750, AA494171, AA099288, AW139363, AW135801, AA101983, AF196329, AF213457, AF241220, NM006678.1, NM004821.1, and N41388.

The invention provides isolated polypeptides or proteins comprising an amino acid sequence that comprises or consists of at least about 10, 15, 20, 25, 30, 50, 75, 100, 125, 150, 175, 200 or more contiguous amino acids of SEQ ID NO:6, or fragments, homologues, derivatives, or variants thereof. In specific embodiments, such polypeptides or proteins exclude polypeptides or proteins encoded by nucleotide sequences of accession nos. D78812, AI337247, AW139572, AW274906, AW139573, AI394041, AI621023, AI86456, AI968134, AI394092, AI681036, AI962750, AA494171, AA099288, AW139363, AW135801, AA101983, AF196329, AF213457, AF241220, NM006678.1, NM004821.1, and N41388.

In preferred embodiments, such fragments, homologues, derivatives or variants of TREM-4 (including TREM-4-alpha and TREM-4-beta) or TREM-5 have a biological activity of a TREM-4 (including TREM-4-alpha and TREM-4-beta) or TREM-5 full-length protein, such as antigenicity, immunogenicity, triggering of proinflammatory chemokines and cytokines, mobilization of cytosolic Ca²⁺, protein tyrosine-phosphorylation, and other activities readily assayable.

In one embodiment, this invention provides isolated nucleic acid molecules which hybridize under stringent or moderately stringent conditions, as defined herein, to a nucleic acid having the sequence of SEQ ID NO:1, 2 or 3, or a complement thereof.

Furthermore, this invention also provides nucleic acid molecules which are suitable for use as primers or hybridization probes for the detection of nucleic acids encoding a polypeptide of the invention.

In one embodiment, the invention provides an isolated nucleic acid molecule which is antisense to the coding strand of a nucleic acid of the invention.

Another aspect of the invention provides vectors, e.g., recombinant expression vectors, comprising a nucleic acid molecule of the invention. Further, the invention also provides host cells containing such a vector or engineered to contain and/or express a nucleic acid molecule of the invention and host cells containing a nucleotide sequence of the invention operably linked to a heterologous promoter. The invention also provides methods for preparing a polypeptide of the invention by a recombinant DNA technology in which the host cells containing a recombinant expression vector encoding a polypeptide of the invention or a nucleotide sequence encoding a polypeptide of the invention operably linked to a heterologous promoter, are cultured, and the polypeptide of the invention produced and isolated.

The invention further provides antibodies that specifically bind a polypeptide of the invention. Such antibodies include, but are not limited to, synthetic, polyclonal, monoclonal, bi-specific, multi-specific, human, humanized, chimeric antibodies, single chain antibodies, Fab fragments, F(ab′)₂ fragments, disulfide-linked Fvs, and fragments containing either a VL or VH domain or even a complementary determining region (CDR) that specifically binds to a polypeptide of the invention.

In one embodiment, the invention provides methods for detecting the presence, activity or expression of a polypeptide of the invention in a biological material, such as cells, blood, saliva, urine, biopsied tissue and so forth and even in vivo. The increased or decreased activity or expression of the polypeptide in a sample relative to a control sample can be determined by contacting the biological material with an agent which can detect directly or indirectly the presence, activity or expression of the polypeptide of the invention.

In another embodiment, an agent modulates the expression of a polypeptide of the invention by modulating transcription, splicing, or translation of an mRNA encoding a polypeptide of the invention. In one embodiment, such an agent is a nucleic acid molecule having a nucleotide sequence that is antisense to all or a portion of the coding strand of an mRNA encoding a polypeptide of the invention. In another embodiment, the agent is double stranded RNA identical to or homologous to all or a portion of a nucleic acid of the invention such that the double stranded RNA mediates RNA.

The invention also provides methods for modulating the activity of a polypeptide of the invention comprising contacting a cell with an agent that modulates (e.g., inhibits or stimulates) the activity or expression of a polypeptide of the invention. In one embodiment, such a modulating agent is an antibody that is specific for a polypeptide of the invention. In another embodiment, the agent is a polypeptide or a fragment of a polypeptide of the invention or a nucleic acid molecule encoding such a polypeptide fragment.

In another aspect, the present invention provides methods for identifying a compound or ligand that binds to or modulates the activity of a polypeptide of the invention. Such a method comprises measuring a biological activity of the polypeptide in the presence and absence of a test compound and identifying test compounds that alter (increase or decrease) the biological activity of the polypeptide. In another aspect, the invention provides a method for identifying a compound that modulates the expression of a polypeptide or nucleic acid of the invention by measuring the expression of the polypeptide or nucleic acid in the presence or absence of the compound.

In one embodiment, the invention provides a fusion protein comprising a bioactive molecule and one or more domains of a polypeptide of the invention or fragment thereof. In particular, the present invention provides fusion proteins comprising a bioactive molecule recombinantly fused or chemically conjugated (including both covalent and non-covalent conjugations) to one or more domains of a polypeptide of the invention or fragments thereof. The domains of the polypeptide include but is not limited to the extracellular region, signal peptide, Ig-SF domain, transmembrane domain, consensus sequence, and cytoplasmic tail.

The present invention also provides methods for treating a subject having a disorder which is characterized by aberrant activity of a polypeptide of the invention or aberrant expression of a nucleic acid of the invention. The present invention further provides methods of contraception, particularly for use in males, e.g., to inhibit or reduce spermatogenesis by administering an agent which is a modulator of the activity of a polypeptide of the invention or a modulator of the expression of a nucleic acid of the invention to the subject. In one embodiment, such modulator is a polypeptide of the present invention or fragments thereof. In another embodiment, such modulator is a nucleic acid of the invention (e.g., gene therapy). In another embodiment, the modulator may be an antibody which is specific to a polypeptide of the invention.

Furthermore, the invention provides a pharmaceutical composition comprising a polypeptide or nucleic acid molecule of the present invention or an antibody or fragment thereof specific to a polypeptide of the invention.

The invention further provides a kit containing a polypeptide or nucleic acid molecule of the present invention or an antibody or fragments thereof specific to a polypeptide of the invention.

### 3.1 Definitions

The term “immunoglobulin superfamily” or “Ig-SF” refers to a group of cell membrane proteins having a common structure similar to an immunoglobulin constant region (C1-type and C2-type) or variable region (V-type). The prototype of V-type domains are the variable domains of immunoglobulins and T-cell receptors. V-type immunoglobulin domains are larger than C1 and C2 domains. Some proteins carry many such domains and others few.

The term “triggering receptor expressed on myeloid cells” or “TREM” refers to a group of activating receptors which are selectively expressed on different types of myeloid cells, such as monocytes, macrophages, dendritic cells (DCs), and neutrophils, and may have a predominant role in inflammatory responses. TREMs are primarily transmembrane glycoproteins with a Ig-type fold in their extracellular domain and, hence, belong to the Ig-SF. These receptors contain a short intracellular domain, but lack docking motifs for signaling mediators and require adapter proteins, such as DAP12, for cell activation.

The term “myeloid cells” as used herein refers to a series of bone marrow-derived cell lineages including granulocytes (neutrophils, eosinophils, and basophils), monocytes, macrophages, and mast cells. Furthermore, peripheral blood dendritic cells of myeloid origin, and dendritic cells and macrophages derived in vitro from monocytes in the presence of appropriate culture conditions, are also included.

The term “homologue,” especially “TREM homologue” as used herein refers to any member of a series of peptides or nucleic acid molecules having a common biological activity, including antigenicity/immunogenicity and inflammation regulatory activity, and/or structural domain and having sufficient amino acid or nucleotide sequence identity as defined herein. TREM homologues can be from either the same or different species of animals.

The term “variant” as used herein refers either to a naturally occurring allelic variation of a given peptide or a recombinantly prepared variation of a given peptide or protein in which one or more amino acid residues have been modified by amino acid substitution, addition, or deletion. Preferably, the term “variant” has common biological activity, etc.

The term “derivative” as used herein refers to a variation of given peptide or protein that are otherwise modified, i.e., by covalent attachment of any type of molecule, preferably having bioactivity, to the peptide or protein, including non-naturally occurring amino acids. Preferably, the term “derivative” has common biological activity, etc.

An “isolated” or “purified” peptide or protein is substantially free of cellular material or other contaminating proteins from the cell or tissue source from which the protein is derived, or substantially free of chemical precursors or other chemicals when chemically synthesized. The language “substantially free of cellular material” includes preparations of a polypeptide/protein in which the polypeptide/protein is separated from cellular components of the cells from which it is isolated or recombinantly produced. Thus, a polypeptide/protein that is substantially free of cellular material includes preparations of the polypeptide/protein having less than about 30%, 20%, 10%, 5%, 2.5%, or 1% (by dry weight) of contaminating protein. When the polypeptide/protein is recombinantly produced, it is also preferably substantially free of culture medium, i.e., culture medium represents less than about 20%, 10%, or 5% of the volume of the protein preparation. When polypeptide/protein is produced by chemical synthesis, it is preferably substantially free of chemical precursors or other chemicals, i.e., it is separated from chemical precursors or other chemicals which are involved in the synthesis of the protein. Accordingly, such preparations of the polypeptide/protein have less than about 30%, 20%, 10%, 5% (by dry weight) of chemical precursors or compounds other than polypeptide/protein fragment of interest. In a preferred embodiment of the present invention, polypeptides/proteins are isolated or purified.

An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment of the invention, nucleic acid molecules encoding polypeptides/proteins of the invention are isolated or purified. However, “isolated” nucleic acid molecule does not include a clone that is part of a cDNA library population.

An “isolated” or “purified” antibody or fragment thereof is substantially free of cellular material or other contaminating proteins from the cell or tissue source from which the protein is derived, or substantially free of chemical precursors or other chemicals when chemically synthesized. The language “substantially free of cellular material” includes preparations of an antibody or antibody fragment in which the antibody or antibody fragment is separated from cellular components of the cells from which it is isolated or recombinantly produced. Thus, an antibody or antibody fragment that is substantially free of cellular material includes preparations of antibody or antibody fragment having less than about 30%, 20%, 10%, or 5% (by dry weight) of heterologous protein (also referred to herein as a “contaminating protein”). When the antibody or antibody fragment is recombinantly produced, it is also preferably substantially free of culture medium, i.e., culture medium represents less than about 20%, 10%, or 5% of the volume of the protein preparation. When the antibody or antibody fragment is produced by chemical synthesis, it is preferably substantially free of chemical precursors or other chemicals, i.e., it is separated from chemical precursors or other chemicals which are involved in the synthesis of the protein. Accordingly such preparations of the antibody or antibody fragment have less than about 30%, 20%, 10%, 5% (by dry weight) of chemical precursors or compounds other than the antibody or antibody fragment of interest. In a preferred embodiment, antibodies of the invention or fragments thereof are isolated or purified.

Other abbreviations used herein are: SIRPβ1, signal regulatory protein β1; HA, hemagglutinin; TNP, 2,4,6-trinitrophenyl; MCP, monocyte chemoattractant protein; PLC-γ, phospholipase C-γ; DC, dendritic cell; MPO, myeloperoxidase; ITAM, immunoreceptor tyrosine-based activation motif; ERK, extracellular signal-related kinase; mAb, monoclonal antibody.

The names of amino acids referred to herein are abbreviated either with three-letter or one-letter symbols.

### 4. DESCRIPTION OF THE FIGURES

FIG. 1 shows the nucleotide sequence of TREM-4-alpha (SEQ ID NO:1).

FIG. 2 shows the nucleotide sequence of TREM-4-beta (SEQ ID NO:2).

FIG. 3 shows the nucleotide sequence of TREM-5 (SEQ ID NO:3).

FIG. 4 shows the predicted amino acid sequence of TREM-4-alpha (SEQ ID NO:4). The signal peptide is indicated in lower-case letters. The potential N-glycosylation site is indicated in bold and italic. The cysteines potentially involved in generating the intrachain disulfide bridge of the 1 g-SF V-type fold are marked in bold and are shown in the context of their flanking consensus sequences. The predicted transmembrane domain is underlined and the charged arginine residue is marked in bold.

FIG. 5 shows the predicted amino acid sequence of TREM-4-beta (SEQ ID NO:5). The signal peptide is indicated in lower-case letters. The potential N-glycosylation site is indicated in bold and italic. The cysteines potentially involved in generating the intrachain disulfide bridge of the Ig-SF V-type fold are marked in bold and are shown in the context of their flanking consensus sequences. The predicted transmembrane domain is underlined and the charged arginine residue is marked in bold.

FIG. 6 shows the predicted amino acid sequences of TREM-5 (SEQ ID NO:6). The signal peptide is indicated in lower-case letters. The potential N-glycosylation site is indicated in bold and italic. The cysteines potentially involved in generating the intrachain disulfide bridge of the Ig-SF V-type fold are marked in bold and are shown in the context of their flanking consensus sequences. The predicted transmembrane domain is underlined and the charged arginine residue is marked in bold.

FIG. 7A shows the alignment of TREM-4 (SEQ ID NO:7), TREM-5 (SEQ ID NO:8), CMRF-35 (SEQ ID NO:9), polymeric Ig-receptor (PIGR) (SEQ ID NO:10), NKp44 (SEQ ID NO:11), TREM-2 (SEQ ID NO:12), TREM-1 (SEQ ID NO:13), and TREM-3 (SEQ ID NO:14) Ig-SF V-type domain amino acid sequences. Only one domain of the five PIGR Ig-SF V-type domains is included. All sequences are from human polypetides with the exception of mouse TREM-3. Amino acids identical in at least 5 out of 8 sequences are included in dark gray boxes. Amino acids similar in at least 5 out of 8 sequences are included in light gray boxes. The alignment was generated by Clustal method. Gaps (dashes) were introduced to maximize homologies.

FIG. 7B shows a phylogenetic tree of the amino acid sequences extracellular Ig-SF V-type domains of TREM-4 (SEQ ID NO:7), TREM-5 (SEQ ID NO:8), CMRF-35 (SEQ ID NO:9), polymeric Ig-receptor (PIGR) (SEQ ID NO:10), NKp44 (SEQ ID NO:11), TREM-2 (SEQ ID NO:12), TREM-1 (SEQ ID NO:13), and TREM-3 (SEQ ID NO:14) based upon amino acid sequence similarity. The cytoplasmic Tyr-Asn-Met-Phe motif is indicated in bold and italic.

FIG. 8 shows a cytogenetic map of human chromosome 17 and the localization of human TREM-4 on human chromosome 17q21 is indicated by a square.

FIG. 9A shows a cytogenetic map of human chromosome 17 and the localization of human TREM-5 on human chromosome 17q25 is indicated by a square.

FIG. 9B shows a genetic map of the CMRF-35-TREM-5 cluster. CMRF-35, CMRF-35H and TREM-5 are indicated by arrows. Transcripts homologous to CMRF-35 are indicated in black. Transcripts homologous to TREM-5 are indicated in gray. The contig on the left is accessible on NCBI human genome sequence database under the accession number: NT_{—}010672.8|Hs17_{—}10829.

FIG. 10 shows the expression of TREM-4 transcripts in human tissues. TREM-4 is selectively expressed in testis, heart, skeletal muscle and placenta. The migration of standard size markers is shown on the right.

FIGS. 11A-E show the expression of TREM-4 in capillaries. TREM-4 is expressed in the endothelium of capillaries of subcutaneous adipose tissue (FIG. 11A), lymph nodes (FIG. 11B), thymus (FIGS. 11C-D) and liver (FIG. 11E). Hepatic sinusoids are also TREM-4-positive (FIG. 11E). FIG. 11D is a higher magnification of FIG. 11C.

FIGS. 12A-D show the expression of TREM-4 in testis. TREM-4 is expressed in spermatogenic cells of seminiferous tubules.

FIGS. 12B and 12D are higher magnifications of seminiferous tubules. In FIGS. 12A and 12B, nuclei are counterstained with Mayers hematoxylin.

FIGS. 13A-B show the expression of TREM-4 in testis is restricted togerm cells. In testis, TREM-4 is not expressed in Sertoli cells (FIG. 13A, arrows) or Leydig cells (FIG. 13B, arrows).

FIG. 14 shows expression of TREM-4 mRNA in heart tissues. TREM-4 mRNA expression in different heart tissues and in control human DNA after profiling on a MTE™ array consisting of a total of 75 tissue-specific poly A⁺ RNAs directly spotted on a nylon membrane. After hybridization with ³²P-labelled TREM-4 specific probe, membranes were exposed for 2 days in a phosphoimager. Bars represent optical density of spots (counts of the pixels within the object).

FIG. 15 shows expression of TREM-4 mRNA and protein in heart. Panel A) TREM-4 mRNA was quantified by real time PCR as described in Methods. Levels of TREM-4 mRNA normalized to β-actin are shown. H: total heart; SM: smooth muscle. Panel B) Western blot analysis of proteins specifically immunoprecipitated from heart of donor 1 (lane 1 and 2), heart of donor 2 (lane 3 and 4), supernatants of J558 cells transfected with huTREM-4/IgG (lane 5 and 6), non-transfected J558 cells (lane 7), huTREM-4-transfected J558 cells (lane 8), and T cells (lane 9). Arrows indicate the differentially expressed bands: the band at 50 kD represents soluble huTREM-4/IgG, the band at 30 kD represents surface expressed huTREM-4. The thick arrow on the right at approximately 25 kD indicates a non-specific band also present upon immunoprecipitation with control mouse IgG1.

FIG. 16 shows expression of TREM-4 ligand in A549 cells. Histogram plots represent expression of TREM-4 ligand in A549 cells that were stained with huTREM-4/IgG and analyzed by flow cytometry (empty histograms). Filled histograms represent staining with control IgG. Surface expression (panel A) and intracellular expression (panel B) of TREM-4 ligand are reported. Cells were stained with different concentrations of huTREM-4/IgG: 1, 0.5, 0.1 μg. Histogram plots represent TREM-4 ligand expression at the cell surface (panel C) and intracellular (panel D).

FIG. 17 shows expression of TREM-4 ligand on epithelial cell lines. Histograms plots represent surface expression of TREM-4 ligand in three different epithelial cell lines: A-549, DU-145, ECV-304 obtained from ATCC. Cells that were stained with huTREM-4/IgG and analyzed by flow cytometry (dotted lines). Black lines represent staining with control IgG.

FIG. 18 shows expression of TREM-5 mRNA. TREM-5 mRNA was quantified by real time PCR as described in Methods. Levels of TREM-5 mRNA normalized to β-actin are shown. Panel A): Heart: normal heart; sm: normal smooth muscle; bm: bone marrow placenta: normal placenta; lung: normal lung; kidney: normal kidney; pbmc a: peripheral blood mononuclear cells from donor a; pbmc b: peripheral blood mononuclear cells from donor b. Panel b): imm mddc: monocyte-derived immature dendritic cells; mddc sac: monocyte derived dendritic cells stimulated with SAC; mddc CD40L: monocyte derived dendritic cells stimulated via CD40 ligation; mono a: peripheral monocytes from donor a; mono b: peripheral monocytes from donor b; pDC: resting plasmacytoid dendritic cells; mDC: resting myeloid dendritic cells.

### 5. DETAILED DESCRIPTION OF THE INVENTION

This invention relates generally to new activating receptors of the Ig super-family expressed on human myeloid cells, called TREM (triggering receptor expressed on myeloid cells) which are involved in inflammatory responses. Specifically, this invention relates to TREM-4 and its homologue, TREM-5.

### 5.1 Human TREM-4 and TREM-5

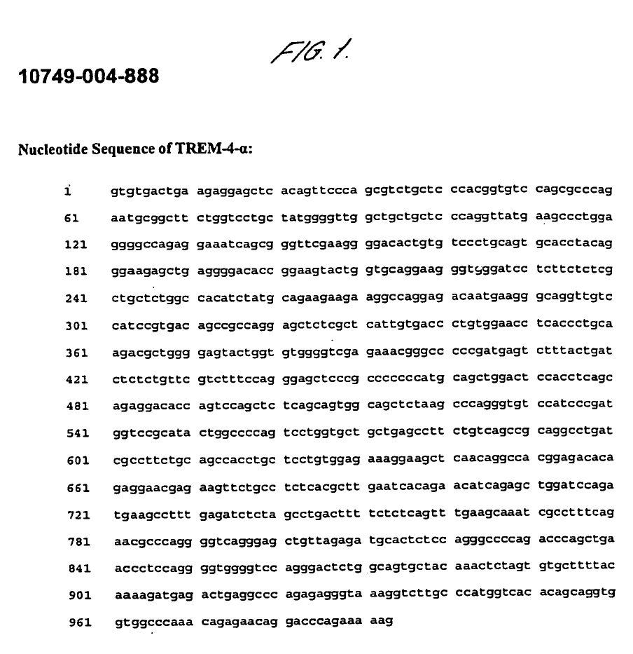

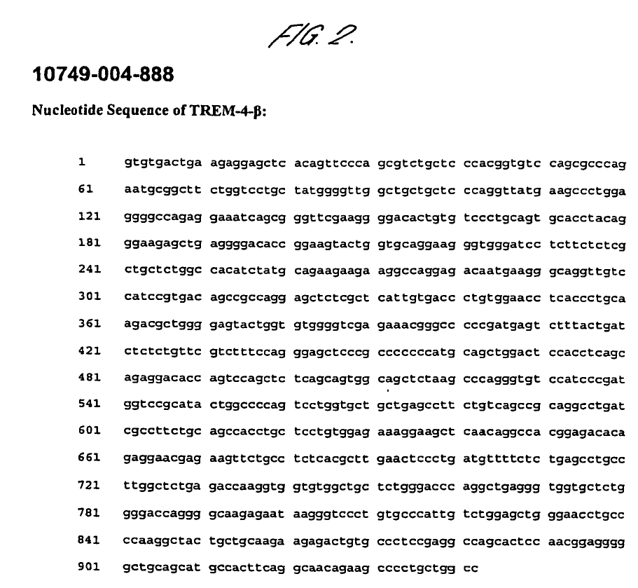

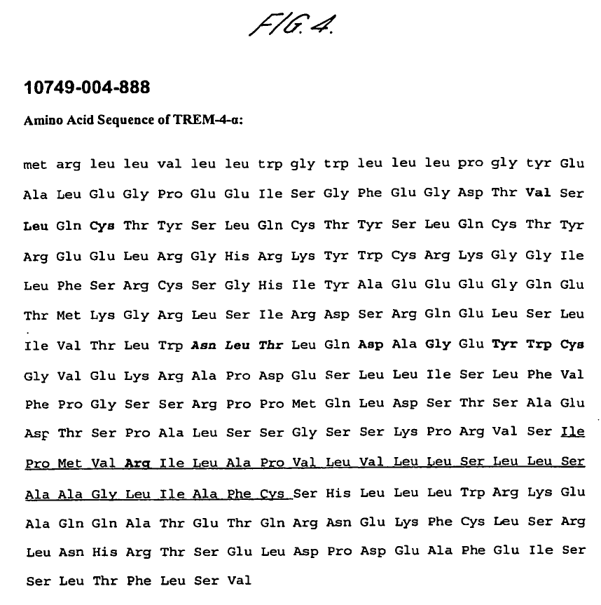

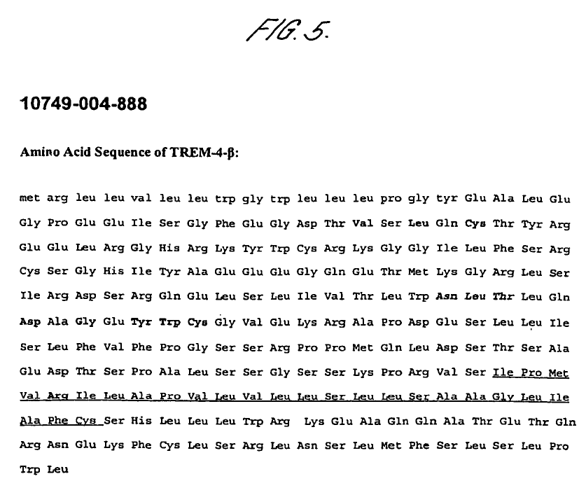

cDNAs encoding TREM-4 were discovered by its homology to NKp44. The human TREM-4-alpha cDNA is 993-nucleotide long (FIG. 1; SEQ ID NO:1) and the human TREM-4-beta cDNA is 942-nucleotide long (FIG. 2; SEQ ID NO:2). The open reading frame of TREM-4-alpha is nucleotides 62 to 763 of SEQ ID NO:1, which encodes a transmembrane protein comprising the 233 amino acid sequence shown in FIG. 4 (SEQ ID NO:4). The open reading frame of TREM-4-beta is nucleotides 62 to 730 of SEQ ID NO:2, which encodes a transmembrane protein comprising the 222 amino acid sequence shown in FIG. 5 (SEQ ID NO:5). TREM-4-alpha and TREM-4-beta represent alternatively spliced forms of the same transcript.

TREM-4 is a novel Ig-SF cell surface molecule that is expressed in the endothelium of certain capillaries and in the testis and heart. TREM-4 has been implicated in inflammation and neoplastic transformation. TREM-4 is strongly expressed in the capillaries of subcutaneous adipose tissue, lymph nodes, thymus, as well as in liver sinusoid endothelium cells, but not in the capillaries of dermis, lung and placenta, the endothelium of arteries, arterioles, veins, venules and lymphatic vessels. TREM-4 may be differentially expressed in different types of testicular germ cell tumors (seminomas and nonseminomatous germ cell tumors, e.g., NSGCTs). TREM-4 has utility in the regulation of inflammation, neoplastic transformation, myeloid cells, atherosclerosis, tumorigenesis, spermatogenesis, and microcirculation. Further utility for TREM-4 is therefore present in the treatment of various heart disorders for example, myocardial necrosis, cardiomegaly, cardiac failure, myocardial infarction, ischemia, coronary artery disease, atherosclerosis or angina pectoris.

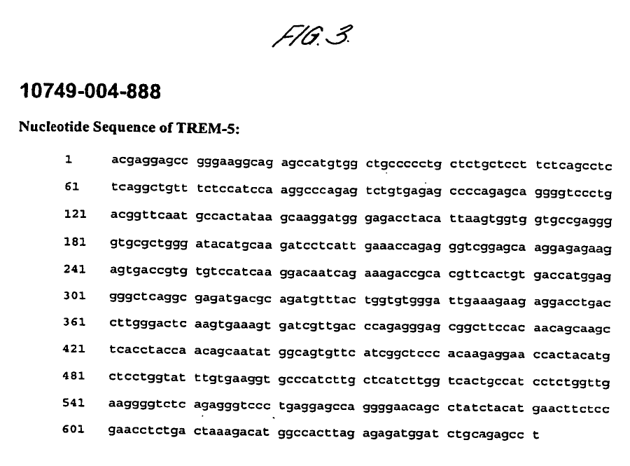

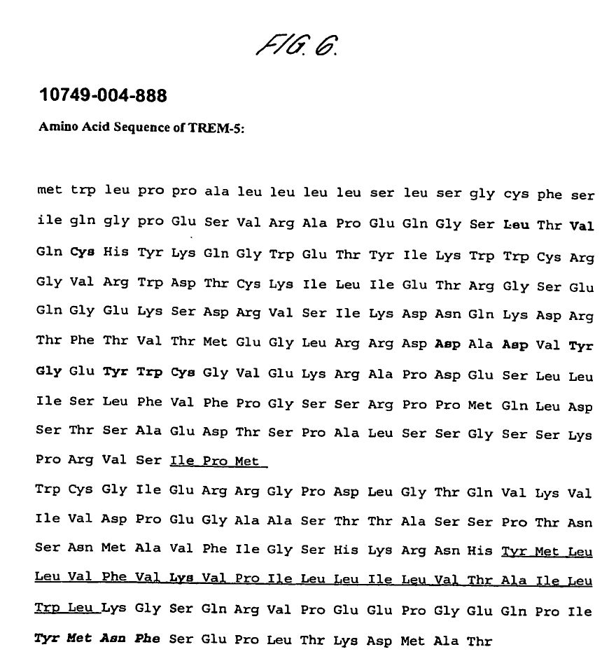

In addition to TREM-4, the present inventors also cloned a novel cDNA encoding a TREM-4-homologue, called TREM-5. The cDNA encoding human TREM-5 is 651-nucleotides long (FIG. 3; SEQ ID NO:3) and the open reading frame of TREM-5 comprises nucleotides 25 to 630 of SEQ ID NO:3, which encode a transmembrane protein comprising the 201 amino acid sequence shown in FIG. 6 (SEQ ID NO:6). TREM-5 is selectively expressed in bone marrow-derived population of leukocytes in particular on dendritic cells and may be modulated in certain conditions, such as cell activation or inflammation. TREM-5 contains a charged lysine residue in the transmembrane region which is reminiscent of that of activating NK cell and myeloid cell receptors that pair with the transmembrane adapter proteins DAP12. Through association with DAP12, TREM-5 may be involved in the pathogenesis of skin diseases such as atopic dermatitis, familial psoriasis, and epidermodysplasia verruciformis. TREM-5 has utility in the regulation of dermal responses to pathogens. Thus, distinct TREM receptors may regulate acute and chronic inflammatory responses, allowing myeloid cells to mount distinct types of responses to different antigens.

Both TREM-4 and TREM-5 display some sequence homology with activating NK cell receptors, such as NKp44 (Cantoni, C., C. Bottino, M. Vitale, A. Pessino, R. Augugliaro, A. Malaspina, S. Parolini, L. Moretta, A. Moretta, and R. Biassoni, 1999, NKp44, a triggering receptor involved in tumor cell lysis by activated human natural killer cells, is a novel member of the immunoglobulin superfamily. J. Exp. Med. 189:787). All of these molecules display a single V-type Ig-like extracellular domain and associate with DAP12 to induce activation. In addition, they are encoded by genes on human chromosome 17. Thus, this chromosome may contain a gene cluster encoding structurally related receptors that activate cell types involved in different innate responses.

As shown in FIG. 4 (SEQ ID NO:4), the predicted amino acid sequence of TREM-4-alpha starts with a hydrophobic signal peptide at amino acid residues 1 to 16 of SEQ ID NO:4 (SEQ ID NO:15) followed by an extracellular region composed of a single Ig-SF domain, encompassing amino acid residues 17 to 233 of SEQ ID NO:4 (SEQ ID NO:16), which contain one potential N-glycosylation site at amino acid residues 96 to 99 of SEQ ID NO:4 (Asn-Leu-Thr-Leu; SEQ ID NO:17), and the consensus sequence Asp-Xaa-Gly-Xaa-Tyr-Xaa-Cys at positions 101 to 107 of SEQ ID NO:4 (SEQ ID NO:18), characteristic of the intrachain disulfide bridge of the 1 g-SF V-type fold. The putative transmembrane domain starts from amino acid residues 164 to 190 of SEQ ID NO:4 (SEQ ID NO:19) and contains a charged arginine residue at position 162. Its cytoplasmic tail consists of 50 amino acid residues at position 184 to 233 of SEQ ID NO:4 (SEQ ID NO:20) and appears to contain no signaling motifs.

As shown in FIG. 5 (SEQ ID NO:5), the predicted amino acid sequence of TREM-4-beta starts with a hydrophobic signal peptide at amino acid residues 1 to 16 of SEQ ID NO:5 (SEQ ID NO:21) followed by an extracellular region composed of a single Ig-SF domain, encompassing amino acid residues 17 to 222 of SEQ ID NO:5 (SEQ ID NO:22), which contain one potential N-glycosylation sites at amino acid residues 96 to 99 of SEQ ID NO:5 (Asn-Leu-Thr-Leu; SEQ ID NO:23), and the consensus sequence Asp-Xaa-Gly-Xaa-Tyr-Xaa-Cys at positions 101-107 of SEQ ID NO:5 (SEQ ID NO:24), characteristic of the intrachain disulfide bridge of the Ig-SF V-type fold. The putative transmembrane domain starts from amino acid residues 164 to 190 of SEQ ID NO:5 (SEQ ID NO:25) and contains a charged arginine residue at position 162. Its cytoplasmic tail consists of 39 amino acid residues at positions 184 to 222 of SEQ ID NO:5 (SEQ ID NO:26) and appears to contain no signaling motifs.

As shown in FIG. 6 (SEQ ID NO:6), the predicted amino acid sequence of TREM-5 starts with a hydrophobic signal peptide at amino acid residues 1 to 21 of SEQ ID NO:6 (SEQ ID NO:27) followed by an extracellular region composed of a single Ig-SF domain, encompassing amino acid residues 22 to 201 of SEQ ID NO:6 (SEQ ID NO:28), which contain no potential N-glycosylation sites but does contain the consensus sequence Asp-Xaa-Gly-Xaa-Tyr-Xaa-Cys at positions 98 to 104 of SEQ ID NO:6 (SEQ ID NO:29), characteristic of the intrachain disulfide bridge of the Ig-SF V-type fold. The putative transmembrane domain starts from amino acid residues 151 to 183 of SEQ ID NO:6 (SEQ ID NO:30) and contains a charged lysine residue at position 158. Its cytoplasmic tail consists of 29 amino acid residues at position 173 to 201 of SEQ ID NO:6 (SEQ ID NO:31) and appears to contain no signaling motifs.

A “signal sequence” or “signal peptide” as used herein refers to a peptide of at least about 10 to 40 amino acid residues which occurs at the N-terminus of secretory or membrane-bound proteins and contains at least about 50-75% hydrophobic amino acid residues such as alanine, leucine, isoleucine, phenylalanine, proline, tyrosine, tryptophan, or valine. A signal sequence serves to direct a protein containing such a sequence to a lipid bilayer. A signal sequence is usually cleaved during the maturation process of the protein. Thus, the invention also includes the domains and the mature protein resulting from cleavage of such a signal peptide.

Accordingly, a mature TREM comprises one or more of the following domains: (1) an extracellular domain which contains at least one 1 g-SF domain; (2) a transmembrane domain; and (3) a cytoplasmic domain.

Thus, in one embodiment, a polypeptide of the invention comprises the amino acid sequence of SEQ ID NO:4, 5 or 6. In another embodiment, a polypeptide of the invention is a mature polypeptide which does not contain a signal peptide and comprises amino acid residues 17 to 233 of SEQ ID NO:4 (SEQ ID NO:16) or amino acid residues 17 to 222 of SEQ ID NO:5 (SEQ ID NO:22) or amino acid residues 22 to 201 of SEQ ID NO:6 (SEQ ID NO:28). In another aspect, a polypeptide of the invention comprises the amino acid sequence of SEQ ID NO:4, 5 or 6 except that amino acid residues 1 to 16 of SEQ ID NO:4 (SEQ ID NO:15) or amino acid residues 1 to 16 of SEQ ID NO:5 (SEQ ID NO:21) or amino acid residues 1 to 21 of SEQ ID NO:6 (SEQ ID NO:27) are replaced by a heterologous signal peptide by genetic engineering.

Yet, in another embodiment, a polypeptide of the invention comprises an extracellular domain comprising amino acid residues 17 to 233 of SEQ ID NO:4 (SEQ ID NO:16) or amino acid residues 17 to 222 of SEQ ID NO:5 (SEQ ID NO:22) or amino acid residues 22 to 201 of SEQ ID NO:6 (SEQ ID NO:28). In another embodiment, a polypeptide of the invention comprises a transmembrane domain comprising amino acid residues 164 to 190 of SEQ ID NO:4 (SEQ ID NO:19) or amino acid residues 164 to 190 of SEQ ID NO:5 (SEQ ID NO:25) or amino acid residues 151 to 183 of SEQ ID NO:6 (SEQ ID NO:30).

Further, a polypeptide of the invention comprises a cytoplasmic domain comprising amino acid residues 184 to 233 of SEQ ID NO:4 (SEQ ID NO:20) or amino acid residues 184 to 222 of SEQ ID NO:5 (SEQ ID NO:26) or amino acid residues 173 to 201 of SEQ ID NO:6 (SEQ ID NO:31).

In preferred embodiments, a polypeptide of the invention comprises a fragment of SEQ ID NO:4, 5 or 6 which exhibits at least one structural and/or functional feature of TREM-4 or TREM-5, wherein said functional feature includes a capability of eliciting a specific immune response, such as producing anti-TREM-4-alpha or anti-TREM-4-beta or anti-TREM-5 antibodies or ability to be immunospecifically bound by anti-TREM-4-alpha or anti-TREM-4-beta or anti-TREM-5 antibodies.

Included within the present invention is an isolated nucleic acid molecule that encodes a polypeptide of the invention having the amino acid sequence of SEQ ID NO:4, 5, 6, 15, 16, 18, 19, 20, 21, 22, 24, 25, 26, 27, 28, 29, 30, 31, or a complement thereof. The nucleic acid molecules of the invention include the entire or a portion (of at least 5, 10, 15, 20, 25, 50, 75, 100, 150, 200, 250, 300, 350, 400, 450, 500, 550, 600, 650 or more contiguous nucleotides) of the nucleotide sequence of SEQ ID NO:1, 2 or 3, or a complement thereof, respectively, which corresponds to the nucleotide sequence encoding the amino acid sequence of SEQ ID NO:4, 5 or 6, respectively. Furthermore, because of the genetic code degeneracy, the invention also includes nucleic acid molecules that are different from SEQ ID NO:1, 2, 3, 32, 33, 34, 35, 36, 37, 38, 39, 40, 41, 42, 43, 44, 45, or 46 but encode the amino acid sequence of SEQ ID NO:4, 5, 6, 15, 16, 18, 19, 20, 21, 22, 24, 25, 26, 27, 28, 29, 30, or 31.

### 5.2 Homologues, Variants, and Derivatives of TREM-4 and TREM-5

In addition to the nucleic acid molecules and polypeptides described above, nucleic acid molecules or polypeptides of the invention also encompass those nucleic acid molecules and polypeptides having a common biological activity and/or structural domain and having sufficient nucleotide sequence or amino acid identity (homologues) as defined herein. These homologues can be from either the same or different species of animal, preferably from mammals, more preferably from rodents, such as mouse and rat, and most preferably from human. Preferably, they exhibit at least one structural and/or functional feature of TREM-4 or TREM-5, including antigenicity/immunogenicity.

Homologues of the nucleic acid molecules of the invention can be isolated based on their close identity to the human nucleic acid molecules disclosed herein, by standard hybridization techniques under stringent or moderately stringent conditions, as defined herein below, using the human cDNA of the invention or a portion thereof as a hybridization probe.

Accordingly, the invention also includes an isolated nucleic acid molecule being at least 25, 50, 100, 200, 300, 400, 500, 600, 650 or more contiguous nucleotides in length and hybridizing under stringent or moderately stringent conditions to the nucleic acid molecule comprising the nucleotide sequence of NO:1, 2, 3, 32, 33, 34, 35, 36, 37, 38, 39, 40, 41, 42, 43, 44, 45, or 46, or a complement thereof.

The term “under stringent condition” refers to hybridization and washing conditions under which nucleotide sequences having at least 60%, preferably 65%, more preferably 70%, most preferably 75% identity to each other remain hybridized to each other. The term “moderately stringent condition” refers to hybridization and washing conditions under which nucleotide sequences having at least 40%, preferably 45%, more preferably 50%, most preferably 55% identity to each other remain hybridized to each other. Such hybridization conditions are described in, for example but not limited to, Current Protocols in Molecular Biology, John Wiley & Sons, N.Y. (1989), 6.3.1-6.3.6, and Basic Methods in Molecular Biology, Elsevier Science Publishing Co., Inc., N.Y. (1986), pp. 75-78, and 84-87, and are well known to those skilled in the art. A preferred, non-limiting example of stringent hybridization conditions is hybridization in 6× sodium chloride/sodium citrate (SSC) at about 45° C. followed by one or more washes in 0.2×SSC, 0.1% SDS at about 50-65° C. A preferred, non-limiting example of moderately stringent conditions is hybridization in 6×SSC at about 42° C. followed by one or more washes in 0.2× S.C., 0.1% SDS at about 45-55° C.

In another aspect, an isolated nucleic acid molecule of the invention encodes a variant of a polypeptide of the invention in which the amino acid sequences have been modified by genetic engineering in order to either enhance or reduce biological activities of the polypeptides, or change the local structures thereof without significantly altering the biological activities. In one aspect, these variants can act as either agonists or as antagonists. An agonist can retain substantially the same or a portion of the biological activities of the polypeptides of the invention and an antagonist can inhibit one or more of the activities of the polypeptides of the invention. Such modifications include amino acid substitution, deletion, and/or insertion. Amino acid modifications can be made by any method known in the art and various methods are available to and routine for those skilled in the art.

For example, mutagenesis may be performed in accordance with any of the techniques known in the art including, but not limited to, synthesizing an oligonucleotide having one or more modifications within the sequence of a given polypeptide to be modified. Site-specific mutagenesis can be conducted using specific oligonucleotide sequences which encode the nucleotide sequence containing the desired mutations in addition to a sufficient number of adjacent nucleotides in the polypeptide. Such oligonucleotides can serve as primers which can form a stable duplex on both sides of the deletion junction being traversed. Typically, a primer of about 17 to about 75 nucleotides or more in length is preferred, with about 10 to about 25 or more residues on both sides of the junction of the sequence being altered. A number of such primers introducing a variety of different mutations at one or more positions may be used to generated a library of mutants.

The technique of site-specific mutagenesis is well known in the art, as described in various publications (e.g., Kunkel et al., Methods Enzymol., 154:367-82, 1987, which is hereby incorporated by reference in its entirety). In general, site-directed mutagenesis is performed by first obtaining a single-stranded vector or melting apart of two strands of a double stranded vector which includes within its sequence a DNA sequence which encodes the desired peptide. An oligonucleotide primer bearing the desired mutated sequence is prepared, generally synthetically. This primer is then annealed with the single-stranded vector, and subjected to DNA polymerizing enzymes such as T7 DNA polymerase, in order to complete the synthesis of the mutation-bearing strand. Thus, a heteroduplex is formed wherein one strand encodes the original non-mutated sequence and the second strand bears the desired mutation. This heteroduplex vector is then used to transform or transfect appropriate cells, such as E. coli cells, and clones are selected which include recombinant vectors bearing the mutated sequence arrangement. As will be appreciated, the technique typically employs a phage vector which exists in both a single stranded and double stranded form. Typical vectors useful in site-directed mutagenesis include vectors such as the M13 phage. These phage are readily commercially available and their use is generally well known to those skilled in the art. Double stranded plasmids are also routinely employed in site directed mutagenesis which eliminates the step of transferring the gene of interest from a plasmid to a phage.

Alternatively, the use of PCR™ with commercially available thermostable enzymes such as Taq DNA polymerase can be used to incorporate a mutagenic oligonucleotide primer into an amplified DNA fragment that can then be cloned into an appropriate cloning or expression vector. See, e.g., Tomic et al., Nucleic Acids Res., 18(6):1656, 1987, and Upender et al., Biotechniques, 18(1):29-30, 32, 1995, for PCR™-mediated mutagenesis procedures, which are hereby incorporated in their entireties. PCR™ employing a thermostable ligase in addition to a thermostable polymerase can also be used to incorporate a phosphorylated mutagenic oligonucleotide into an amplified DNA fragment that can then be cloned into an appropriate cloning or expression vector (see e.g., Michael, Biotechniques, 16(3):410-2, 1994, which is hereby incorporated by reference in its entirety).

Other methods known to those skilled in art of producing sequence variants of a given polypeptide or a fragment thereof (e.g., an extracellular-domain, transmembrane-domain, and cytoplasmic-domain fragments) can be used. For example, recombinant vectors encoding the amino acid sequence of the polypeptide or a fragment thereof can be treated with mutagenic agents, such as hydroxylamine, to obtain sequence variants.

Preferably, the amino acid residues to be modified are surface exposed residues. Additionally, in making amino acid substitutions, preferably the amino acid residue to be substituted is a conservative amino acid substitution, for example, a polar residue is substituted with a polar residue, a hydrophilic residue with a hydrophilic residue, hydrophobic residue with a hydrophobic residue, a positively charged residue with a positively charged residue, or a negatively charged residue with a negatively charged residue. Moreover, preferably, the amino acid residue to be modified is not highly or completely conserved across species and/or is critical to maintain the biological activities of the protein.

Accordingly, included in the scope of the invention are nucleic acid molecules encoding a polypeptide of the invention that contains amino acid modifications that are not critical to activity. Thus, an isolated nucleic acid molecule of the invention includes a nucleotide sequence encoding a polypeptide having an amino acid sequence that is at least about 40%, 45%, 50%, 55%, 60%, 65%, 70%, 75%, 80%, 85%, 90%, 95%, or 98% identical to the amino acid sequence of SEQ ID NO:4, 5, 6, 15, 16, 18, 19, 20, 21, 22, 24, 25, 26, 27, 28, 29, 30, or 31.

Furthermore, the invention also encompasses derivatives of the polypeptides of the invention. For example, but not by way of limitation, derivatives can include peptides or proteins that have been modified e.g., by glycosylation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to, specific chemical cleavage, acetylation, formylation, etc. Additionally, the derivative can contain one or more non-classical amino acids.

In another aspect, the invention further includes antisense nucleic acid molecules which are complementary to an entire or partial sense nucleic acid encoding a polypeptide of the invention (e.g., a coding strand of cDNA or a mRNA). The antisense nucleic acid molecules can also be complementary to non-coding region of the nucleic acid which will not be translated. The antisense nucleic acid molecules of the invention can be administered to a subject so that they hybridize with cellular mRNA or genomic DNA which encodes a polypeptide of the invention. This blocks the transcription and/or translation of the target sequence and, thereby inhibits expression of the polypeptide. An antisense nucleic acid can be about 5, 10, 15, 20, 25, 30, 35, 40, 45, or 50 nucleotides long or longer and can be prepared by chemical synthesis and enzymatic ligation reactions using methods well known in the art. For, example, an antisense nucleic acid can be chemically synthesized using naturally occurring nucleotides or variously modified nucleotides designed to increase the biological stability of the molecules or to increase the physical stability of the duplex formed between the antisense and sense nucleic acids; for example, phosphorothioate derivatives and acridine substituted nucleotides can be used.

Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Left. 215:327-330).

The invention also encompasses ribozymes. Ribozymes are catalytic RNA molecules with ribonudease activity which are capable of cleaving a single-stranded nucleic acid, such as an mRNA, to which they have a complementary region. Thus, ribozymes, such as hammerhead ribozymes (described in Haselhoff and Gerlach, 1988, Nature, 334:585-591) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA. For example, a derivative of a Tetrahymena L-19 IVS RNA can be constructed in which the nucleotide sequence of the active site is complementary to the nucleotide sequence to be cleaved (Cech et al., U.S. Pat. No. 4,987,071; and Cech et al., U.S. Pat. No. 5,116,742). Alternatively, an mRNA encoding a polypeptide of the invention disclosed herein can be used to select a catalytic RNA having a specific ribonuclease activity from a pool of RNA molecules. See, e.g., Bartel and Szostak, 1993, Science, 261:1411-1418.

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene, 1991, Anticancer Drug Des., 6(6):569-84; Helene, 1992, Ann. N.Y. Acad. Sci., 660:27-36; and Maher, 1992, Bioassays, 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al., 1996, Bioorganic & Medicinal Chemistry, 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al., supra; Perry-O'Keefe et al., 1996, Proc. Natl. Acad. Sci. USA, 93:14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup, supra); or as probes or primers for DNA sequence and hybridization (Hyrup, supra; Perry-O'Keefe et al., supra).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, supra). The synthesis of PNA-DNA chimeras can be performed as described in Hyrup, supra, and Finn et al. (1996, Nucleic Acids Res., 24(17):3357-63). For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al., 1989, Nucleic Acids Res., 17:5973-88). PNA monomers are then coupled in a stepwise manner to produce a chimeric molecule with a 5′ PNA segment and a 3′ DNA segment (Finn et al., 1996, Nucleic Acids Res., 24(17):3357-63). Alternatively, chimeric molecules can be synthesized with a 5′ DNA segment and a 3′ PNA segment (Peterser et al., 1975, Bioorganic Med. Chem. Lett., 5:1119-11124).

In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA, 86:6553-6556; Lemaitre et al., 1987, Proc. Natl. Acad. Sci. USA, 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques, 6:958-976) or intercalating agents (see, e.g., Zon, 1988, Pharm. Res., 5:539-549). To this end, the oligonucleotide can be conjugated to another molecule, e.g., a peptide, hybridization triggered cross-linking agent, transport agent, hybridization-triggered cleavage agent, etc.

### 5.3 Recombinant Expression Vectors and Host Cells

The present invention also provides vectors, preferably expression vectors, containing a nucleic acid encoding a polypeptide of the invention or a portion thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain expression vectors, are capable of directing the expression of genes to which they are operably linked. In general, expression vectors of utility in recombinant DNA techniques are often in the form of plasmids (vectors). However, the invention also includes other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. In other words, the recombinant expression vectors include one or more regulatory sequences, preferably heterologous to the nucleic acid of the invention, which are selected on the basis of the host cells to be used for expression and are operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

A variety of host-vector systems can be utilized in the present invention to express the protein-coding sequence. These include but are not limited to bacteria transformed with bacteriophage, DNA, plasmid DNA, or cosmid DNA; microorganisms such as yeast containing yeast vectors; insect cell systems infected with virus (e.g., baculovirus); or mammalian cell systems infected with virus (e.g., vaccinia virus, adenovirus, etc.). The expression elements of vectors vary in their strengths and specificities. Depending on the host-vector system utilized, any one of a number of suitable transcription and translation elements can be used. Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Fusion proteins comprising a polypeptide of the invention are further discussed in section 5.4 below.

Examples of suitable inducible non-fusion E. coli expression vectors include pTrc (Amann et al., 1988, Gene 69:301-315) and pET 11d (Studier et al., Gene Expression Technology: Methods in Enzymology, 185, Academic Press, San Diego, Calif., 1990, pp. 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident A prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif., 1990, pp. 119-128). Another strategy is to alter the nucleic acid sequence of the nucleic acid to be inserted into an expression vector so that the individual codons for each amino acid are those preferentially utilized in E. coli (Wada et al., 1992, Nucleic Acids Res., 20:2111-2118). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO J., 6:229-234), pMFa (Kurjan and Herskowitz, 1982, Cell, 30:933-943), pJRY88 (Schultz et al., 1987, Gene, 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol., 3:2156-2165) and the pVL series (Lucklow and Summers, 1989, Virology, 170:31-39).

In yet another embodiment, a nucleic acid of the invention is expressed in mammalian cells using a mammalian expression vector. Examples of mammalian expression vectors include pCDM8 (Seed, 1987, Nature, 329:840) and pMT2PC (Kaufman et al., 1987, EMBO J., 6:187-195). When used in mammalian cells, the expression vector's control functions are often provided by viral regulatory elements. For example, commonly used promoters are derived from polyoma, adenovirus 2, cytomegalovirus and Simian Virus 40. For other suitable expression systems for both prokaryotic and eukaryotic cells see chapters 16 and 17 of Sambrook et al., (1990, Molecular Cloning, A Laboratory Manual, 2d Ed., Cold Spring Harbor Laboratory, Cold Spring Harbor, N.Y.).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev., 1:268-277), lymphoid-specific promoters (Calame and Eaton, 1988, Adv. Immunol., 43:235-275), in particular promoters of T cell receptors (Winoto and Baltimore, 1989, EMBO J., 8:729-733) and immunoglobulins (Banerji et al., 1983, Cell, 33:729-740; Queen and Baltimore, 1983, Cell, 33:741-748), neuron-specific promoters (e.g., the neurofilament promoter; Byrne and Ruddle, 1989, Proc. Natl. Acad. Sci. USA, 86:5473-5477), pancreas-specific promoters (Edlund et al., 1985, Science, 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science, 249:374-379) and the α-fetoprotein promoter (Campes and Tilghman, 1989, Genes Dev., 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1), 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not be identical to the parent cell, but are still included within the scope of the term as used herein.

A host cell can be any prokaryotic (e.g., E. coli) or eukaryotic cell (e.g., insect cells, yeast or mammalian cells). A host cell strain can be selected which modulates the expression of the inserted sequences, or modifies and processes the gene product in the specific fashion desired. Expression from certain promoters can be elevated in the presence of certain inducers; thus, expression of the genetically engineered polypeptide/protein may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 3T3, WI38, and in particular, neuronal cell lines such as, for example, SK—N-AS, SK—N-FI, SK—N-DZ human neuroblastomas (Sugimoto et al., 1984, J. Natl. Cancer Inst., 73:51-57), SK—N—SH human neuroblastoma (Biochim. Biophys. Acta, 1982, 704:450-460), Daoy human cerebellar medulloblastoma (He et al., 1992, Cancer Res., 52:1144-1148) DBTRG-05MG glioblastoma cells (Kruse et al., 1992, In Vitro Cell. Dev. Biol., 28A:609-614), IMR-32 human neuroblastoma (Cancer Res., 1970, 30:2110-2118), 1321N1 human astrocytoma (Proc. Natl. Acad. Sci. USA, 1977, 74:4816), MOG-G-CCM human astrocytoma (Br. J. Cancer, 1984, 49:269), U87MG human glioblastoma-astrocytoma (Acta Pathol. Microbiol. Scand., 1968, 74:465-486), AI72 human glioblastoma (Olopade et al., 1992, Cancer Res., 52:2523-2529), C6 rat glioma cells (Benda et al., 1968, Science, 161:370-371), Neuro-2a mouse neuroblastoma (Proc. Natl. Acad. Sci. USA, 1970, 65:129-136), NB41A3 mouse neuroblastoma (Proc. Natl. Acad. Sci. USA, 1962, 48:1184-1190), SCP sheep choroid plexus (Bolin et al., 1994, J. Virol. Methods, 48:211-221), G355-5, PG-4 Cat normal astrocyte (Haapala et al., 1985, J. Virol., 53:827-833), Mpf ferret brain (Trowbridge et al., 1982, In Vitro, 18:952-960), and normal cell lines such as, for example, CTX TNA2 rat normal cortex brain (Radany et al., 1992, Proc. Natl. Acad. Sci. USA, 89:6467-6471) such as, for example, CRL7030 and Hs578Bst. Furthermore, different vector/host expression systems may effect processing reactions to different degrees.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al., supra, and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker is generally introduced into the host cells along with the gene of interest. A number of selection systems may be used, including but not limited to the herpes simplex virus thymidine kinase (Wigler, et al., 1977, Cell, 11:223), hypoxanthine-guanine phosphoribosyltransferase (Szybalska & Szybalski, 1962, Proc. Natl. Acad. Sci. USA, 48:2026), and adenine phosphoribosyltransferase (Lowy, et al., 1980, Cell, 22:817) genes can be employed in tk⁻, hgprt⁻ or aprt⁻ cells, respectively. Also, antimetabolite resistance can be used as the basis of selection for dhfr, which confers resistance to methotrexate (Wigler, et al., 1980, Natl. Acad. Sci. USA, 77:3567; O'Hare, et al., 1981, Proc. Natl. Acad. Sci. USA, 78:1527); gpt, which confers resistance to mycophenolic acid (Mulligan & Berg, 1981, Proc. Natl. Acad. Sci. USA, 78:2072); neo, which confers resistance to the aminoglycoside G-418 (Colberre-Garapin, et al., 1981, J. Mol. Biol., 150:1); and hygro, which confers resistance to hygromycin (Santerre, et al., 1984, Gene, 30:147) genes.

In another embodiment, the expression characteristics of an endogenous gene sequence (e.g., TREM-4, including TREM-4-alpha and TREM-4-beta; TREM-5) within a cell, cell line or cloned microorganism may be modified by inserting a DNA regulatory element heterologous to the endogenous gene of interest into the genome of a cell, stable cell line or cloned microorganism such that the inserted regulatory element is operatively linked with the endogenous gene and controls, modulates or activates the endogenous gene. For example, endogenous TREM-4 and TREM-5 which are expressed only at very low levels in a cell or cell line, may be activated by inserting a regulatory element which is capable of promoting the expression of a normally expressed gene product in that cell or cell line. Alternatively, endogenous TREM-4 and TREM-5 genes which are normally “transcriptionally silent”, i.e., TREM-4 and TREM-5 genes which are normally not expressed, may be activated by insertion of a promiscuous regulatory element that works across cell types.

A heterologous regulatory element may be inserted into a stable cell line or cloned microorganism, such that it is operatively linked with and activates expression of endogenous TREM-4 and TREM-5 genes, using techniques, such as targeted homologous recombination, which are well known to those skilled in the art, and described, for example, in Chappel, U.S. Pat. No. 5,272,071; PCT publication No. WO 91/06667 (published May 16, 1991).

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of the invention, into which a recombinant expression vector encoding a polypeptide of the invention has been introduced, in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a sequence encoding a polypeptide of the invention has been introduced. Such host cells can then be used to create non-human transgenic animals in which exogenous sequences encoding a polypeptide of the invention have been introduced into their genome or homologous recombinant animals in which endogenous sequences encoding a polypeptide of the invention have been altered. Such animals are useful for studying the function and/or activity of the polypeptide and for identifying and/or evaluating modulators of polypeptide activity. In addition to particular gene expression and/or polypeptide expression phenotypes, the transgenic animals of the invention can exhibit any of the phenotypes (e.g., processes, disorder symptoms and/or disorders associated with the gene expression). As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a polypeptide of the invention or a homologue thereof into the male pronuclei of a fertilized oocyte, for example, by microinjection or retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986) and Wakayama et al., (1999), Proc. Natl. Acad. Sci. USA, 96:14984-14989. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al., 1992, Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed., IRL, Oxford, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, 1991, Current Opinion in Bio/Technology, 2:823-829, and in PCT Publication Nos. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the crelloxP recombinase system, see, e.g., Lakso et al., 1992, Proc. Natl. Aced. Sci. USA, 89:6232-6236. Another example of a recombinase system is the FLP recombinase system of Saccharomyces cerevisiae (O'Gorman et al., 1991, Science, 251:1351-1355). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al., 1997, Nature, 385:810-813, and PCT Publication Nos. WO 97/07668 and WO 97/07669.

### 5.4 Fusion Proteins

The present invention further encompasses fusion proteins in which the polypeptides of the invention or fragments thereof, are recombinantly fused or chemically conjugated (including both covalent and non-covalent conjugations) to heterologous polypeptides (i.e., an unrelated polypeptide; or portion thereof, preferably at least 10, at least 20, at least 30, at least 40, at least 50, at least 60, at least 70, at least 80, at least 90 or at least 100 amino acids of the polypeptide) to generate fusion proteins. The fusion does not necessarily need to be direct, but may occur through linker sequences.

In one example, a fusion protein in which a polypeptide of the invention or a fragment thereof can be fused to sequences derived from various types of immunoglobulins. For example, a polypeptide of the invention can be fused to a constant region (e.g., hinge, CH2, and CH3 domains) of human IgG1 or IgM molecule so as to make the fused polypeptides or fragments thereof more soluble and stable in vivo. Such fusion proteins can be used as an immunogen for the production of specific antibodies which recognize the polypeptides of the invention or fragments thereof. In another embodiment, such fusion proteins can be administered to a subject so as to inhibit interactions between a ligand and its receptors in vivo. Such inhibition of the interaction will block or suppress signal transduction which triggers certain cellular responses. Sections 6.1.6 and 6.2.4 below describe the product of fusion proteins between the extracellular portion of TREM-4 and TREM-5, and the constant domain of human IgG1 (called TREM-4-huIgG1 and TREM-5-huIgG1, respectively).

In one aspect, the fusion protein comprises a polypeptide of the invention which is fused to a heterologous signal sequence at its N-terminus. For example, the signal sequence naturally found in the polypeptide of the invention can be replaced by a signal sequence which is derived from a heterologous origin. Various signal sequences are commercially available. For example, the secretory sequences of melittin and human placental alkaline phosphatase (Stratagene; La Jolla, Calif.) are available as eukaryotic heterologous signal sequences. As examples of prokaryotic heterologous signal sequences, the phoA secretory signal (Sambrook et al., supra; and Current Protocols in Molecular Biology, Ausubel et al., eds., John Wiley & Sons, 1992) and the protein A secretory signal (Pharmacia Biotech; Piscataway, N.J.) can be listed. Another example is the gp67 secretory sequence of the baculovirus envelope protein (Current Protocols in Molecular Biology, Ausubel et al., eds., John Wiley & Sons, 1992).

In another embodiment, a polypeptide of the invention can be fused to tag sequences, e.g., a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA, 86:821-824, for instance, hexa-histidine provides for convenient purification of the fusion protein. Other examples of peptide tags are the hemagglutinin “HA” tag, which corresponds to an epitope derived from the influenza hemagglutinin protein (Wilson et al., 1984, Cell, 37:767) and the “flag” tag (Knappik et al., 1994, Biotechniques, 17(4):754-761). These tags are especially useful for purification of recombinantly produced polypeptides of the invention.

Fusion proteins can be produced by standard recombinant DNA techniques or by protein synthetic techniques, e.g., by use of a peptide synthesizer. For example, a nucleic acid molecule encoding a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992).

The nucleotide sequence coding for a fusion protein can be inserted into an appropriate expression vector, i.e., a vector which contains the necessary elements for the transcription and translation of the inserted protein-coding sequence. Various host-vector systems and selection systems are available as described in section 5.3.

In a specific embodiment, the expression of a fusion protein is regulated by a constitutive promoter. In another embodiment, the expression of a fusion protein is regulated by an inducible promoter. In accordance with these embodiments, the promoter may be a tissue-specific promoter.

Expression vectors containing inserts of a gene encoding a fusion protein can be identified by three general approaches: (a) nucleic acid hybridization, (b) presence or absence of “marker” gene functions, and (c) expression of inserted sequences. In the first approach, the presence of a gene encoding a fusion protein in an expression vector can be detected by nucleic acid hybridization using probes comprising sequences that are homologous to an inserted gene encoding the fusion protein. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a nucleotide sequence encoding a fusion protein in the vector. For example, if the nucleotide sequence encoding the fusion protein is inserted within the marker gene sequence of the vector, recombinants containing the gene encoding the fusion protein insert can be identified by the absence of the marker gene function. In the third approach, recombinant expression vectors can be identified by assaying the gene product (i.e., fusion protein) expressed by the recombinant. Such assays can be based, for example, on the physical or functional properties of the fusion protein in in vitro assay systems, e.g., binding with anti-fusion protein antibody.

For long-term, high-yield production of recombinant proteins, stable expression is preferred. For example, cell lines which stably express the fusion protein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines that express the differentially expressed or pathway gene protein. Such engineered cell lines may be particularly useful in screening and evaluation of compounds that affect the endogenous activity of the differentially expressed or pathway gene protein.

Once a fusion protein of the invention has been produced by recombinant expression, it may be purified by any method known in the art for purification of a protein, for example, by chromatography (e.g., ion exchange, affinity, particularly by affinity for the specific antibody, and sizing column chromatography), centrifugation, differential solubility, or by any other standard technique for the purification of proteins.

### 5.5 Preparation of Antibodies

Antibodies which specifically recognize a polypeptide of the invention or fragments thereof can be used for various diagnostic and therapeutic purposes. For example, in one specific embodiment, an antibody which is specific for TREM-4 or TREM-5 can be used for various in vitro detection assays, including enzyme-linked immunosorbent assays (ELISA), radioimmunoassays, Western blot, Flow Cytometry analysis, immunohistochemical analysis, and so forth for the detection of TREM-4 or TREM-5 molecules or fragments thereof in biological samples, such as blood, serum, plasma, urine, saliva, tissues, cells, etc., as well as for in vivo detection of these molecules for diagnostic purposes. For example, an anti-TREM-4 antibody can be used in immunohistochemical analysis of pathological tissue specimens to differentiate local and systemic inflammations caused by different types of agents. Since TREM-4 is strongly expressed in the presence of certain bacterial and fungal products, such as LPS, detection of TREM-4 in the inflamed tissue specimens would suggest a bacterial and/or fungal origin of the inflammation.

In another specific embodiment, an anti-TREM-4 or anti-TREM-5 antibody which acts as an antagonist against TREM-4 or TREM-5 (i.e., inhibiting TREM-4 or TREM-5 activities), respectively, on myeloid cells may be used as a therapeutic agent for reducing systemic and/or local inflammatory responses triggered by causative agents (e.g., bacteria and fungi). Such antibodies can block the binding of ligands to these receptors and, thereby, prevent subsequent signal transduction and inflammatory responses from occurring. Examples of local inflammations include pulmonitis, pleuritis, impetigo, abscesses, sinovitis, arthritis, etc. and systemic inflammations include meningitis, peritonitis, sepsis, etc. Thus, these antibodies are useful in modulating inflammatory responses mediated by TREM-4 and/or TREM-5.

In another specific embodiment, anti-TREM-5 antibody may be used as an adjuvant to facilitate the migration of DCs (dendritic cells) from the periphery to the lymph nodes by cross-linking TREM-5 on DCs and upregulating CCR7 expression by DCs.

In another specific embodiment, anti-TREM-4 antibodies, including antigen-binding fragments thereof, can be used to inhibit spermatogenesis, e.g., as a contraceptive.

In a further embodiment, anti-TREM-4 antibodies, including antigen-binding fragments thereof, can be used to treat various heart disorders, for example, but not limited to, myocardial necrosis, cardiomegaly, cardiac failure, myocardial infarction, ischemia, coronary artery disease, atherosclerosis or angina pectoris.

Other diagnostic, therapeutic, or prophylactic uses of antibodies specific for the polypeptides of the invention will be further discussed below.

Antibodies specific for the polypeptides of the invention may be generated by any suitable method known in the art. Polyclonal antibodies to an antigen-of-interest can be produced by various procedures well known in the art. For example, an antigen derived from the polypeptide of the invention can be administered to various host animals including, but not limited to, rabbits, mice, rats, etc., to induce the production of antisera containing polyclonal antibodies specific for the antigen. Various adjuvants may be used to increase the immunological response, depending on the host species, and include but are not limited to, Freund's (complete and incomplete) adjuvant, mineral gels such as aluminum hydroxide, surface active substances such as lysolecithin, pluronic polyols, polyanions, peptides, oil emulsions, keyhole limpet hemocyanins, dinitrophenol, and potentially useful adjuvants for humans such as BCG (Bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas, pp. 563-681 (Elsevier, N.Y., 1981) (both of which are incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In a non-limiting example, mice can be immunized with an antigen of interest or a cell expressing such an antigen. Once an immune response is detected, e.g., antibodies specific for the antigen are detected in the mouse serum, the mouse spleen is harvested and splenocytes isolated. The splenocytes are then fused by well known techniques to any suitable myeloma cells. Hybridomas are selected and cloned by limiting dilution. The hybridoma clones are then assayed by methods known in the art for cells that secrete antibodies capable of binding the antigen. Ascites fluid, which generally contains high levels of antibodies, can be generated by inoculating mice intraperitoneally with positive hybridoma clones.

Antibody fragments which recognize specific epitopes may be generated by known techniques. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the complete light chain, and the variable region, the CH1 region and the hinge region of the heavy chain.

The antibodies of the invention or fragments thereof can be also produced by any method known in the art for the synthesis of antibodies, in particular, by chemical synthesis or preferably, by recombinant expression techniques.

The nucleotide sequence encoding an antibody may be obtained from any information available to those skilled in the art (Le., from Genbank, the literature, or by routine cloning). If a clone containing a nucleic acid encoding a particular antibody or an epitope-binding fragment thereof is not available, but the sequence of the antibody molecule or epitope-binding fragment thereof is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., supra; and Ausubel et al., eds., 1998, Current Protocols in Molecular Biology, John Wiley & Sons, NY, which are both incorporated by reference herein in their entireties), to generate antibodies having a different amino acid sequence by, for example, introducing amino acid substitutions, deletions, and/or insertions into the epitope-binding domain regions of the antibodies or any portion of antibodies which may enhance or reduce biological activities of the antibodies.

Recombinant expression of an antibody requires construction of an expression vector containing a nucleotide sequence that encodes the antibody. Once a nucleotide sequence encoding an antibody molecule or a heavy or light chain of an antibody, or portion thereof has been obtained, the vector for the production of the antibody molecule may be produced by recombinant DNA technology using techniques well known in the art as discussed in the previous sections. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The nucleotide sequence encoding the heavy-chain variable region, light-chain variable region, both the heavy-chain and light-chain variable regions, an epitope-binding fragment of the heavy- and/or light-chain variable region, or one or more complementarity determining regions (CDRs) of an antibody may be cloned into such a vector for expression. Thus-prepared expression vector can be then introduced into appropriate host cells for the expression of the antibody. Accordingly, the invention includes host cells containing a polynucleotide encoding an antibody specific for the polypeptides of the invention or fragments thereof.

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides or different selectable markers to ensure maintenance of both plasmids. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature, 322:52, 1986; and Kohler, Proc. Natl. Acad. Sci. USA, 77:2 197, 1980). The coding sequences for the heavy and light chains may comprise cDNA or genomic DNA.

In another embodiment, antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains, such as Fab and Fv or disulfide-bond stabilized Fv, expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage, including fd and M13. The antigen binding domains are expressed as a recombinantly fused protein to either the phage gene III or gene VIII protein. Examples of phage display methods that can be used to make the immunoglobulins, or fragments thereof, of the present invention include those disclosed in Brinkman et al., J. Immunol. Methods, 182:41-50, 1995; Ames et al., J. Immunol. Methods, 184:177-186, 1995; Kettleborough et al., Eur. J. Immunol., 24:952-958, 1994; Persic et al., Gene, 187:9-18, 1997; Burton et al., Advances in Immunology, 57:191-280, 1994; PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired fragments, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques, 12(6):864-869, 1992; and Sawai et al., AJRI, 34:26-34, 1995; and Better et al., Science, 240:1041-1043, 1988 (each of which is incorporated by reference in its entirety). Examples of techniques which can be used to produce single-chain Fvs and antibodies include those described in U.S. Pat. Nos. 4,946,778 and 5,258,498; Huston et al., Methods in Enzymology, 203:46-88, 1991; Shu et al., PNAS, 90:7995-7999, 1993; and Skerra et al., Science, 240:1038-1040, 1988.

Once an antibody molecule of the invention has been produced by any methods described above, it may then be purified by any method known in the art for purification of an immunoglobulin molecule, for example, by chromatography (e.g., ion exchange, affinity, particularly by affinity for the specific antigen after Protein A or Protein G purification, and sizing column chromatography), centrifugation, differential solubility, or by any other standard techniques for the purification of proteins. Further, the antibodies of the present invention or fragments thereof may be fused to heterologous polypeptide sequences described herein or otherwise known in the art to facilitate purification.

For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a constant region derived from a human immunoglobulin. Methods for producing chimeric antibodies are known in the art See e.g., Morrison, Science, 229:1202, 1985; Oi et al., BioTechniques, 4:214 1986; Gillies et al., J. Immunol. Methods, 125:191-202, 1989; U.S. Pat. Nos. 5,807,715; 4,816,567; and 4,816,397, which are incorporated herein by reference in their entireties. Humanized antibodies are antibody molecules from non-human species that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. No. 5,585,089; Riechmann et al., Nature, 332:323, 1988, which are incorporated herein by reference in their entireties. Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Molecular Immunology, 28(415):489498, 1991; Studnicka et al., Protein Engineering, 7(6):805-814, 1994; Roguska et al., Proc Natl. Acad. Sci. USA, 91:969-973, 1994), and chain shuffling (U.S. Pat. No. 5,565,332), all of which are hereby incorporated by reference in their entireties.

Completely human antibodies are particularly desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety.

Human antibodies can also be produced using transgenic mice which are incapable of expressing functional endogenous immunoglobulins, but which can express human immunoglobulin genes. For an overview of this technology for producing human antibodies, see Lonberg and Huszar, Int Rev. Immunol., 13:65-93, 1995. For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties. In addition, companies such as Abgenix, Inc. (Freemont, Calif.), Medarex (NJ) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology, 12:899-903, 1988).

Antibodies fused or conjugated to heterologous polypeptides may be used in in vitro immunoassays and in purification methods (e.g., affinity chromatography) well known in the art. See e.g., PCT publication Number WO 93/21232; EP 439,095; Naramura et al., Immunol. Lett., 39:91-99, 1994; U.S. Pat. No. 5,474,981; Gillies et al., PNAS, 89:1428-1432, 1992; and Fell et al., J. Immunol., 146:2446-2452, 1991, which are incorporated herein by reference in their entireties.

The present invention also encompasses antibodies conjugated to a diagnostic or therapeutic agent. The antibodies can be used diagnostically to, for example, monitor the development or progression of a disease, disorder or infection as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals, and nonradioactive paramagnetic metal ions. The detectable substance may be coupled or conjugated either directly to the antibody or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ¹¹¹In or ^{99m}Tc.

An antibody may be conjugated to a therapeutic moiety such as a cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologues thereof. Therapeutic agents include, but are not limited to, anti-inflammatory agents (e.g., anti-TNFα antibody, e.g., REMICADE® (infliximab) (Centocor, Pa.), IL-1 receptor antagonist, anti-MIF antibody, anti-HMG-1 antibody, and methotrexate), antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an antibody may be conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity.

Antibodies specific for TREM-4 may be useful in treating fertility, in contraceptive methods, in the treatment of tumors, particularly germ cell tumors, as anti-inflammatories or for treatment of various heart disorders for example, but not limited to, myocardial necrosis, cardiomegaly, cardiac failure, myocardial infarction, ischemia, coronary artery disease, atherosclerosis or angina pectoris. Antibodies specific for TREM-5 may be useful for treating or preventing inflammation or in diseases or disorders involving cell activation.

In another embodiment, an antibody specific for TREM-5 (anti-TREM-5 antibody) can be used for an efficient presentation of an antigen of interest, e.g., by DCs to T cells. As TREM-5 is expressed in DCs in peripheral tissues (e.g., skin and mucosa), an anti-TREM-5 antibody coupled with an antigen of interest may effectively and efficiently deliver the antigen to peripheral DCs, which subsequently process and present the antigen to the T cells at lymph nodes. An antigen of interest can be chemically or genetically conjugated to an anti-TREM-5 antibody or, alternatively, the anti-TREM-5 antibody can be genetically engineered to become bispecific for TREM-5 on one arm and for the antigen of interest on the other. This approach may have a great utility in preparing various vaccines.

Techniques for conjugating such therapeutic moieties to antibodies are well known; see, e.g., Amon et al., “Monoclonal Antibodies For Immunotargeting Of Drugs In Cancer Therapy”, in Monoclonal Antibodies And Cancer Therapy, Reisfeld et al., (eds.), 1985, pp. 243-56, Alan R. Liss, Inc.; Hellstrom et al., “Antibodies For Drug Delivery”, in Controlled Drug Delivery (2nd Ed.), Robinson et al. (eds.), 1987, pp. 623-53, Marcel Dekker, Inc.; Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506; “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al., Immunol. Rev., 62:119-58, 1982.

Alternatively, an antibody can be conjugated to a second antibody to form an antibody heteroconjugate as described by Segal in U.S. Pat. No. 4,676,980, which is incorporated herein by reference in its entirety.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### 5.6 Pharmaceutical Compositions

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

A pharmaceutical composition of the invention is formulated to be compatible with its intended route of administration. Examples of routes of administration include parenteral, e.g., intravenous, intradermal, subcutaneous, transdermal (topical), transmucosal, intra-articular, intraperitoneal, and intrapleural, as well as oral, inhalation, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy injectability with a syringe exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. (1997, J. Acquired Immune Deficiency Syndromes and Human Retrovirology, 14:193).

Antibodies or antibodies conjugated to therapeutic moieties can be administered to an individual alone or in combination with cytotoxic factor(s), chemotherapeutic drug(s), anti-inflammatory agents, and/or cytokine(s). If the latter, preferably, the antibodies are administered first and the cytotoxic factor(s), chemotherapeutic drug(s), anti-inflammatory agents, and/or cytokine(s) are administered thereafter within 24 hours. The antibodies and cytotoxic factor(s), chemotherapeutic drug(s) and/or cytokine(s) can be administered by multiple cycles depending upon the clinical response of the patient. Further, the antibodies and cytotoxic factor(s), chemotherapeutic drug(s) and/or cytokine(s) can be administered by the same or separate routes, for example, by intravenous, intranasal or intramuscular administration. Cytotoxic factors include, but are not limited to, TNF-α, TNF-β, IL-1, IFN-γ and IL-2. Chemotherapeutic drugs include, but are not limited to, 5-fluorouracil (5FU), vinblastine, actinomycin D, etoposide, cisplatin, methotrexate and doxorubicin. Cytokines include, but are not limited to, IL-2, IL-3, IL4, IL-5, IL-6, IL-7, IL-8, IL-9, IL-10 and IL-12.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al., 1994, Proc. Natl. Acad. Sci. USA, 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. With regard to gene therapy, see further discussion in section 5.8.3.

The pharmaceutical compositions can be included in a container, pack, or dispenser together with instructions for administration.

### 5.7 Utility and Methods of the Invention

The nucleic acid molecules, proteins, protein homologues, derivatives, variants, and antibodies described herein can be used in one or more of the following methods: a) screening assays; b) detection assays (e.g., chromosomal mapping, tissue typing); c) predictive medicine (e.g., diagnostic assays and prognostic assays); d) methods of treatment (e.g., therapeutic and prophylactic); and e) contraceptive methods. For example, polypeptides of the invention can be used to (i) modulate cellular proliferation; (ii) modulate cellular differentiation; and/or (iii) modulate cellular adhesion. The isolated nucleic acid molecules of the invention can be used to express proteins (e.g., via a recombinant expression vector in a host cell in gene therapy applications), to detect mRNA (e.g., in a biological sample) or a genetic lesion, and to modulate activity of a polypeptide of the invention. In addition, the polypeptides of the invention can be used to screen drugs or compounds which modulate activity or expression of a polypeptide of the invention as well as to treat disorders characterized by insufficient or excessive production of a protein of the invention or production of a form of a protein of the invention which has decreased or aberrant activity compared to the wild type protein. In addition, the antibodies of the invention can be used to detect and isolate a protein of the invention and modulate activity of a protein of the invention.

This invention further pertains to novel agents identified by the above-described screening assays and uses thereof for treatments as described herein.

### 5.7.1 Screening Assays

The invention provides a method for identifying (or screening) modulators, i.e., candidate or test compounds or agents (e.g., peptides, peptidomimetics, small molecules or other drugs) which bind to a polypeptide of the invention or have a stimulatory or inhibitory effect on, for example, expression or activity of a polypeptide of the invention.

In one embodiment, the invention provides assays for screening candidate or test compounds which bind to or modulate the activity of the membrane-bound form of a polypeptide of the invention or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, an assay is a cell-based assay in which a cell which expresses a membrane-bound form of a polypeptide of the invention, or a biologically active portion thereof, on the cell surface is contacted with a test compound and the ability of the test compound to bind to the polypeptide determined. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or a biologically active portion thereof as compared to the known compound.

In another embodiment, an assay is a cell-based assay comprising contacting a cell expressing a membrane-bound form of a polypeptide of the invention, or a biologically active portion thereof, on the cell surface with a test compound and determining the ability of the test compound to modulate (e.g., stimulate or inhibit) the activity of the polypeptide or biologically active portion thereof. Determining the ability of the test compound to modulate the activity of the polypeptide or a biologically active portion thereof can be accomplished, for example, by determining the ability of the polypeptide protein to bind to or interact with a target molecule.

Determining the ability of a polypeptide of the invention to bind to or interact with a target molecule can be accomplished by one of the methods described above for determining direct binding. As used herein, a “target molecule” is a molecule with which a selected polypeptide (e.g., a polypeptide of the invention) binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a polypeptide of the invention or some other polypeptide or protein. For example, a target molecule can be a component of a signal transduction pathway which facilitates transduction of an extracellular signal (e.g., a signal generated by binding of a compound to a polypeptide of the invention) through the cell membrane and into the cell or a second intercellular protein which has catalytic activity or a protein which facilitates the association of downstream signaling molecules with a polypeptide of the invention. Determining the ability of a polypeptide of the invention to bind to or interact with a target molecule can be accomplished by determining the activity of the target molecule. For example, the activity of the target molecule can be determined by detecting induction of a cellular second messenger of the target (e.g., intracellular Ca²⁺, protein tyrosine phosphorylation, phospholipase phosphorylation, etc.), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g., luciferase), or detecting a cellular response, for example, cellular differentiation, or cell proliferation.

In yet another embodiment, an assay of the present invention is a cell-free assay comprising contacting a polypeptide of the invention or biologically active portion thereof with a test compound and determining the ability of the test compound to bind to the polypeptide or biologically active portion thereof. Binding of the test compound to the polypeptide can be determined either directly or indirectly as described above. In a preferred embodiment, the assay includes contacting the polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or biologically active portion thereof as compared to the known compound.

In another embodiment, an assay is a cell-free assay comprising contacting a polypeptide of the invention or biologically active portion thereof with a test compound and determining the ability of the test compound to modulate (e.g., stimulate or inhibit) the activity of the polypeptide or biologically active portion thereof. Determining the ability of the test compound to modulate the activity of the polypeptide can be accomplished, for example, by determining the ability of the polypeptide to bind to a target molecule by one of the methods described above for determining direct binding. In an alternative embodiment, determining the ability of the test compound to modulate the activity of the polypeptide can be accomplished by determining the ability of the polypeptide of the invention to further modulate the target molecule. For example, the catalytic/enzymatic activity of the target molecule on an appropriate substrate can be determined as previously described.

In yet another embodiment, the cell-free assay comprises contacting a polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the polypeptide to preferentially bind to or modulate the activity of a target molecule.

The cell-free assays of the present invention are amenable to use of both a soluble form or the membrane-bound form of a polypeptide of the invention. In the case of cell-free assays comprising the membrane-bound form of the polypeptide, it may be desirable to utilize a solubilizing agent such that the membrane-bound form of the polypeptide is maintained in solution. Examples of such solubilizing agents include non-ionic detergents such as n-octylglucoside, n-dodecylglucoside, n-octylmaltoside, octanoyl-N-methylglucamide, decanoyl-N-methylglucamide, Triton X-100, Triton X-114, Thesit, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either the polypeptide of the invention or its target molecule to facilitate separation of complexed from uncomplexed forms of one or both of the proteins, as well as to accommodate automation of the assay. Binding of a test compound to the polypeptide, or interaction of the polypeptide with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or a polypeptide of the invention, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, either the polypeptide of the invention or its target molecule can be immobilized utilizing conjugation of biotin and streptavidin. Biotinylated polypeptide of the invention or target molecules can be prepared from biotin-NHS(N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptide of the invention or target molecules but which do not interfere with binding of the polypeptide of the invention to its target molecule can be derivatized to the wells of the plate, and unbound target or polypeptide of the invention trapped in the wells by antibody conjugation. Methods for detecting such complexes, in addition to those described above for the GST-immobilized complexes, include immunodetection of complexes using antibodies reactive with the polypeptide of the invention or target molecule, as well as enzyme-linked assays which rely on detecting an enzymatic activity associated with the polypeptide of the invention or target molecule.

In another embodiment, modulators of expression of a polypeptide of the invention are identified in a method in which a cell is contacted with a candidate compound and the expression of the selected mRNA or protein (i.e., the mRNA or protein corresponding to a polypeptide or nucleic acid of the invention) in the cell is determined. The level of expression of the selected mRNA or protein in the presence of the candidate compound is compared to the level of expression of the selected mRNA or protein in the absence of the candidate compound. The candidate compound can then be identified as a modulator of expression of the polypeptide of the invention based on this comparison. For example, when expression of the selected mRNA or protein is greater (statistically significantly greater) in the presence of the candidate compound than in its absence, the candidate compound is identified as a stimulator of the selected mRNA or protein expression. Alternatively, when expression of the selected mRNA or protein is less (statistically significantly less) in the presence of the candidate compound than in its absence, the candidate compound is identified as an inhibitor of the selected mRNA or protein expression. The level of the selected mRNA or protein expression in the cells can be determined by methods described herein.

In yet another aspect of the invention, a polypeptide of the inventions can be used as “bait proteins” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with the polypeptide of the invention and modulate activity of the polypeptide of the invention. Such binding proteins are also likely to be involved in the propagation of signals by the polypeptide of the inventions as, for example, upstream or downstream elements of a signaling pathway involving the polypeptide of the invention.

This invention further pertains to novel agents identified by the above-described screening assays and uses thereof for treatments as described herein.

### 5.7.2 Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

A. Chromosome Mapping

Once the sequence (or a portion of the sequence) of a gene has been isolated, this sequence can be used to map the location of the gene on a chromosome. Accordingly, nucleic acid molecules described herein or fragments thereof, can be used to map the location of the corresponding genes on a chromosome. The mapping of the sequences to chromosomes is an important first step in correlating these sequences with genes associated with disease. The present inventors have mapped the genes encoding TREM-4 and TREM-5 to chromosome 17q21 and 17q25, respectively, in humans.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al., 1987, Nature, 325:783-787.

Moreover, differences in the DNA sequences between individuals affected and unaffected with a disease associated with a gene of the invention can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Furthermore, the nucleic acid sequences disclosed herein can be used to perform searches against “mapping databases”, e.g., BLAST-type search, such that the chromosome position of the gene is identified by sequence homology or identity with known sequence fragments which have been mapped to chromosomes.

A polypeptide and fragments and sequences thereof and antibodies specific thereto can be used to map the location of the gene encoding the polypeptide on a chromosome. This mapping can be carried out by specifically detecting the presence of the polypeptide in members of a panel of somatic cell hybrids between cells of a first species of animal from which the protein originates and cells from a second species of animal and then determining which somatic cell hybrid(s) expresses the polypeptide and noting the chromosome(s) from the first species of animal that it contains. For examples of this technique, see Pajunen et al. (1988) Cytogenet. Cell Genet 47:37-41 and Van Keuren et al. (1986) Hum. Genet. 74:34-40. Alternatively, the presence of the polypeptide in the somatic cell hybrids can be determined by assaying an activity or property of the polypeptide, for example, enzymatic activity, as described in Bordelon-Riser et al. (1979) Somatic Cell Genetics 5:597-613 and Owerbach et al. (1978) Proc. Natl. Acad. Sci. USA 75:5640-5644.

B. Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the nucleic acid sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the noncoding regions. It is estimated that allelic variation between individual humans occurs with a frequency at about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals.

If a panel of reagents from the nucleic acid sequences described herein is used to generate a unique identification database for an individual, those same reagents can later be used to identify tissue from that individual. Using the unique identification database, positive identification of the individual, living or dead, can be made from extremely small tissue samples.

### 5.7.3 Diagnostic Assays

One aspect of the present invention relates to diagnostic assays for determining expression of a polypeptide or nucleic acid of the invention and/or activity of a polypeptide of the invention, in the context of a biological sample (e.g., blood, plasma, serum, cells, tissues) to thereby determine whether an individual is afflicted with a disease or disorder, or is at risk of developing a disorder, associated with aberrant expression or activity of a polypeptide of the invention, such as a proliferative disorder, e.g., psoriasis or cancer, or an angiogenic disorder. The invention also provides for prognostic (or predictive) assays for determining whether an individual is at risk of developing a disorder associated with aberrant expression or activity of a polypeptide of the invention. For example, mutations in a gene of the invention can be assayed in a biological sample. Such assays can be used for prognostic or predictive purpose to thereby prophylactically treat an individual prior to the onset of a disorder characterized by or associated with aberrant expression or activity of a polypeptide of the invention.

An exemplary method for detecting the presence or absence of a polypeptide or nucleic acid of the invention in a biological sample involves obtaining a biological sample from a test subject and contacting the biological sample with a compound or an agent capable of detecting a polypeptide or nucleic acid (e.g., mRNA, genomic DNA) of the invention such that the presence of a polypeptide or nucleic acid of the invention is detected in the biological sample. A preferred agent for detecting mRNA or genomic DNA encoding a polypeptide of the invention is a labeled nucleic acid probe capable of hybridizing to mRNA or genomic DNA encoding a polypeptide of the invention. The nucleic acid probe can be, for example, a full-length cDNA, such as the nucleic acid of SEQ ID NO:1, 2, 3, 32, 33, 34, 35, 36, 37, 38, 39, 40, 41, 42, 43, 44, 45, 46, or a portion thereof, such as an oligonucleotide of at least 15, 20, 25, 30, 50, 100, 250, 500, or more contiguous nucleotides in length and sufficient to specifically hybridize under stringent conditions to a mRNA or genomic DNA encoding a polypeptide of the invention. Other suitable probes for use in the diagnostic assays of the invention are described herein.

A preferred agent for detecting a polypeptide of the invention is an antibody capable of binding to a polypeptide of the invention, preferably an antibody with a detectable label. Antibodies can be polyclonal, or more preferably, monoclonal. An intact antibody, or a fragment thereof (e.g., Fab or F(ab′)₂) can be used. See also the detailed descriptions about antibodies in section 5.5.

The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of a polypeptide of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide of the invention include introducing into a subject a labeled antibody directed against the polypeptide. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a polypeptide of the invention or mRNA or genomic DNA encoding a polypeptide of the invention, such that the presence of the polypeptide or mRNA or genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a polypeptide of the invention as discussed, for example, in sections above relating to uses of the sequences of the invention.

For example, kits can be used to determine if a subject is suffering from or is at increased risk of disorders such as immunological disorders, especially involving inflammatory disorders (e.g., bacterial infection, fungal infection, viral infection, protozoa or other parasitic infection, psoriasis, septicemia, cerebral malaria, inflammatory bowel disease, arthritis, such as rheumatoid arthritis, folliculitis, impetigo, granulomas, lipoid pneumoias, vasculitis, and osteoarthritis), autoimmune disorders (e.g., rheumatoid arthritis, thyroiditis, such as Hashimoto's thyroiditis and Graves' disease, insulin-resistant diabetes, pernicious anemia, Addison's disease, pemphigus, vitiligo, ulcerative colitis, systemic lupus erythematosus (SLE), Sjögren's syndrome, multiple sclerosis, dermatomiositis, mixed connective tissue disease, scleroderma, polymyositis, graft rejection, such as allograft rejection), T cell disorders (e.g., AIDS), allergic inflammatory disorders (e.g., skin and/or mucosal allergies, such as allergic rhinitis, asthma, psoriasis), neurological disorders, eye disorders, embryonic disorders, or any other disorders (e.g., tumors, cancers, leukemia, myeloid diseases, infertility, germ cell tumors, skin diseases, and traumas) which are directly or indirectly associated with aberrant TREM-4 and/or TREM-5 activity and/or expression.

The kit, for example, can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for observing that the tested subject is suffering from or is at risk of developing a disorder associated with aberrant expression of the polypeptide if the amount of the polypeptide or mRNA encoding the polypeptide is above or below a normal level.

For antibody-based kits, the kit can comprise, for example: (1) a first antibody (e.g., attached to a solid support) which binds to a polypeptide of the invention; and, optionally, (2) a second, different antibody which binds to either the polypeptide or the first antibody and is conjugated to a detectable agent.

For oligonucleotide-based kits, the kit can comprise, for example: (1) an oligonucleotide, e.g., a detectably labeled oligonucleotide, which hybridizes to a nucleic acid sequence encoding a polypeptide of the invention or (2) a pair of primers useful for amplifying a nucleic acid molecule encoding a polypeptide of the invention. The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

A. Prognostic Assays

The methods described herein can furthermore be utilized as prognostic assays to identify a subject having or at risk of developing a disorder associated with aberrant expression or activity of a polypeptide of the invention, e.g., an immunologic disorder or other disorders as discussed above.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a gene of the invention, thereby determining if a subject with the lesioned gene is at risk for a disorder characterized aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the misexpression of the gene encoding the polypeptide of the invention. For example, such genetic lesions or mutations can be detected by ascertaining the existence of at least one of the following: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of a the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al., 1988, Science, 241:1077-1080; and Nakazawa et al., 1994, Proc. Natl. Acad. Sci. USA, 91:360-364), the latter of which can be particularly useful for detecting point mutations in a gene (see, e.g., Abravaya et al., 1995, Nucleic Acids Res., 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc. Natl. Acad. Sci. USA, 87:1874-1878), transcriptional amplification system (Kwoh, et al., 1989, Proc. Natl. Acad. Sci. USA, 86:1173-1177), Q-Beta Replicase (Lizardi et al., 1988, Bio/Technology, 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al., 1996, Human Mutation, 7:244-255; Kozal et al., 1996, Nature Medicine, 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert (1977, Proc. Natl. Acad. Sci. USA, 74:560) or Sanger (1977, Proc. Natl. Acad. Sci. USA, 74:5463). It is also contemplated that any of a variety of automated sequencing procedures can be utilized when performing the diagnostic assays (1995, Bio/Techniques, 19:448), including sequencing by mass spectrometry (see, e.g., PCT Publication No. WO 94/16101; Cohen et al., 1996, Adv. Chromatogr., 36:127-162; and Griffin et al., 1993, Appl. Biochem. Biotechnol., 38:147-159).

Other methods for detecting mutations in a selected gene include methods in which protection from cleavage agents is used to detect mismatched bases in RNA/RNA or RNA/DNA heteroduplexes (Myers et al., 1985, Science, 230:1242). In general, the technique of “mismatch cleavage” entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S1 nuclease to digest mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al., 1988, Proc. Natl. Acad. Sci. USA, 85:4397; Saleeba et al., 1992, Methods Enzymol., 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al., 1994, Carcinogenesis, 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al., 1989, Proc. Natl. Acad. Sci. USA, 86:2766; see also Cotton, 1993, Mutat. Res., 285:125-144; Hayashi, 1992, Genet Anal. Tech. Appl., 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al., 1991, Trends Genet, 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a ′GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner, 1987, Biophys. Chem., 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al., 1986, Nature, 324:163); Saiki et al., 1989, Proc. Natl. Acad. Sci. USA, 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al., 1989, Nucleic Acids Res. 17:2437-2448) or at the extreme 3′ end of one primer where, under appropriate conditions, mismatch can prevent or reduce polymerase extension (Prossner, 1993, Tibtech, 11:238). In addition, it may be desirable to introduce a novel restriction site in the region of the mutation to create cleavage-based detection (Gasparini et al., 1992 Mol. Cell Probes, 6:1). It is anticipated that in certain embodiments amplification may also be performed using Taq ligase for amplification (Barany, 1991, Proc. Natl. Acad. Sc. USA, 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a gene encoding a polypeptide of the invention. Furthermore, any cell type or tissue, e.g., preferably peripheral blood leukocytes, in which the polypeptide of the invention is expressed may be utilized in the prognostic assays described herein.

### 5.8 Methods of Treatment

### 5.8.1 Immunoregulatory Effect of TREMs

Inflammatory disorders are generally classified into two types; that is, acute and chronic inflammations. Acute inflammation is triggered by an initiating agent which is often a foreign substance, such as pathogenic organisms (e.g., bacteria, fungi, virus, protozoa and other parasites). The degradation products or toxins released by pathogens may directly cause activation of plasma proteases which leads to a series of inflammatory responses, including vasodilation, increased vascular permeability, recruitment and activation of neutrophils, monocytes, and eosinophils, and production of fever. Furthermore, injured cells can release degradation products which trigger various plasma protease cascades, including complement, kinins, clotting and fibrinolytic proteins, lipid mediators, prostaglandins, leukotrienes, and platelet-activating factor. In addition, expression of proinflammatory cytokines, such as interleukin-1 (IL-1), IL-4, IL-6, IL-8, tumor necrosis factor (TNF) α and β, interferon-γ (IFN-γ), and IL-12, is upregulated and the inflammatory responses are further augmented. The acute phase inflammatory responses are downregulated once the foreign threat is eliminated. Such downregulation is achieved by cell senescence or apoptosis (programmed cell death) which seems to be promoted by certain cytokines, including TNF-α, eicosanoids, IL-10, and antioxidants (Cox et al., 1996, Am J Physiol, 27:L566-L571; Gelrud et al., 1996, Proc Assoc Am Physicians, 108:455-456; Gon et al., 1996, Microbiol Immunol, 40:463-465; Hebert et al., 1996, J. Imunol, 157:3105-3115; Oishi et al., 1997, Scand J Immunol, 45:21-27), and by anti-inflammatory mediators, including IL-4, transforming growth factor-β (TGF-β), IL-10, and IL-13, the latter three being released by macrophages and lymphocytes rather than by granulocytes. However, if the elimination of the foreign substance is incomplete, the inflammatory process persists and chronic inflammation ensues. (See e.g., Rosenberg et al., 1999, Inflammation, in Fundamental Immunology, 4^{th} Ed. W. E. Paul, ed. Lippincott-Raven, Philadelphia p. 1051).

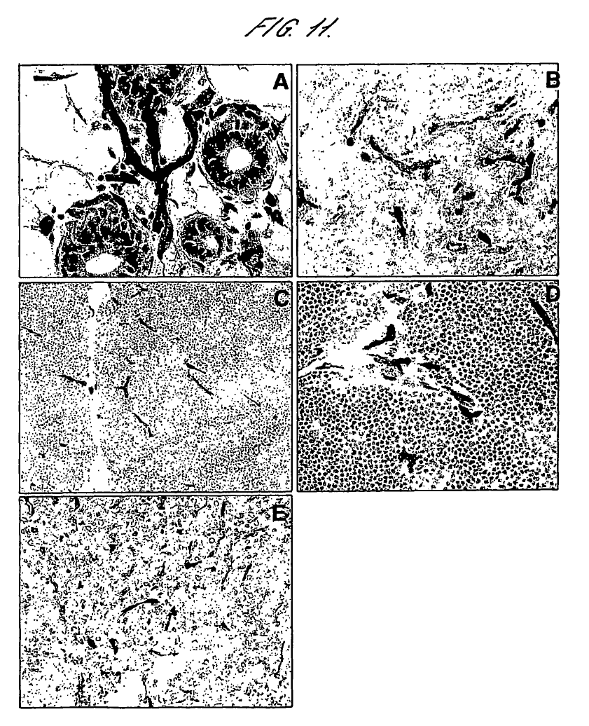

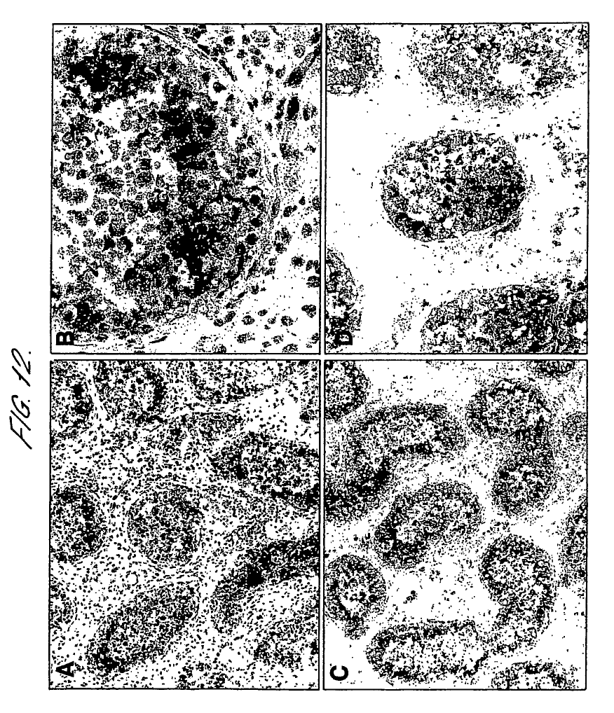

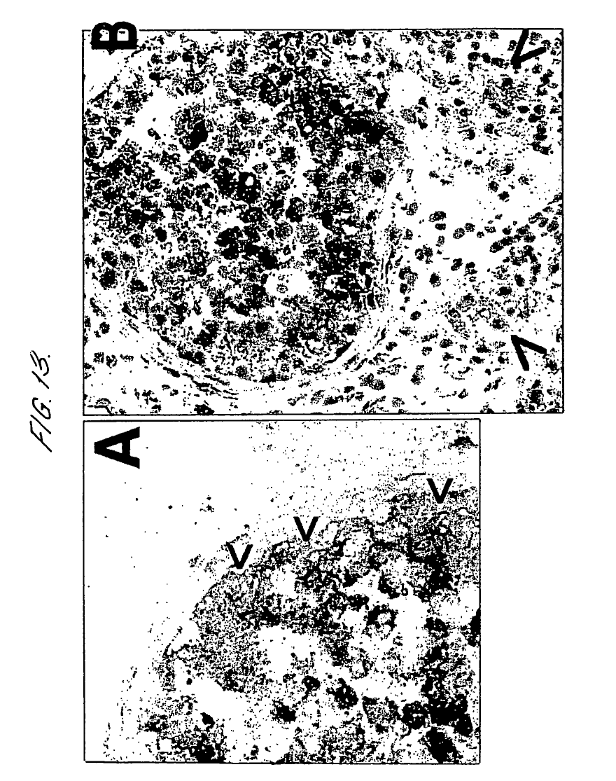

As described in Examples below, the TREMs trigger cell activation, Ca²⁺ mobilization and tyrosine phosphorylation via an associated signal transduction molecule, called DAP12 (Lanier, L. L., 1998, NK cell receptors. Annu. Rev. Immunol., 16:359). Among TREMs, TREM-4 is selectively expressed in the endothelium of capillaries and in the testis. TREM-4 is strongly expressed in the capillaries of subcutaneous adipose tissue (FIG. 11A), lymph nodes (FIG. 11B), thymus (FIGS. 11C-D), as well as in liver sinusoid endothelium cells (FIG. 11E). However, capillaries of dermis, lung and placenta lack expression of TREM-4. Endothelium of arteries, arterioles, veins, venules and lymphatic vessels are TREM-4 negative. In the testis, TREM-4 expression is detectable in the spermatogenic cells of seminiferous tubules (FIGS. 12A-D), whereas Sertoli cells and Leydig cells are negative (FIGS. 13A-B).

As also described in the Examples herein, TREM-4 gene expression and TREM-4 protein is detectable In the heart.

These observations strongly indicate that TREM-4 is involved in inflammatory reactions, neoplastic transformation, microcirculation, and spermatogenesis and thus is a target for treatment of pathogenic inflammatory, neoplastic and microcirculation conditions, heart disorders and diseases, and male infertility.

On the other hand, TREM-5 is predominantly expressed at low levels in a bone marrow-derived population of leukocytes, in particular on dendritic cells, and that TREM-5 may be modulated in certain conditions, such as cell activation or inflammation. TREM-5 contains a charged lysine residue in the transmembrane region which is reminiscent of that of activating NK cell and myeloid cell receptors that pair with the transmembrane adapter proteins DAP12. Through association with DAP12, TREM-5 may be involved in the pathogenesis of skin diseases such as atopic dermatitis, familial psoriasis, and epidermodysplasia verruciformis. TREM-5 has utility in the regulation of dermal responses to pathogens.

Furthermore, DAP12-deficient mice, which are also deficient in TREM-5 function, are more resistant to delayed hypersensitivity reaction (e.g., skin contact allergy) and experimental autoimmune encephalomyelitis (i.e., a mouse model for multiple sclerosis) due to reduced T cell stimulation by DCs (Bakker, A. B., et al., 2000, Immunity 13:345-53; Tomasello, E., et al., 2000, Immunity 13:355-64). Therefore, blocking TREM-5 with, for example, a soluble TREM-5 should reduce adaptive immune responses and protect the host from various immune disorders including autoimmunity and allergies.

Thus, TREM-4 and/or TREM-5 are good targets for preventing and treating various inflammatory disorders and diseases. Accordingly, the present invention provides for both prophylactic and therapeutic methods of treating a subject at risk of (or susceptible to) a disorder or having a disorder associated with aberrant expression or activity of a polypeptide of the invention, as discussed, for example, in sections above relating to uses of the sequences of the invention. For example, disorders characterized by aberrant expression or activity of the polypeptides of the invention include immunological disorders, especially involving inflammatory disorders (e.g., bacterial infection, fungal infection, viral infection, protozoa or other parasitic infection, psoriasis, septicemia, cerebral malaria, inflammatory bowel disease, arthritis, such as rheumatoid arthritis, folliculitis, impetigo, granulomas, lipoid pneumoias, vasculitis, and osteoarthritis), autoimmune disorders (e.g., rheumatoid arthritis, thyroiditis, such as Hashimoto's thyroiditis and Graves' disease, insulin-resistant diabetes, pernicious anemia, Addison's disease, pemphigus, vitiligo, ulcerative colitis, systemic lupus erythematosus (SLE), Sjögren's syndrome, multiple sclerosis, dermatomiositis, mixed connective tissue disease, scleroderma, polymyositis, graft rejection, such as allograft rejection), T cell disorders (e.g., AIDS) and allergic inflammatory disorders (e.g., skin and/or mucosal allergies, such as allergic rhinitis, asthma, psoriasis), neurological disorders, eye disorders, embryonic disorders, seminomas and nonseminomas germ cell tumors, skin diseases (e.g., atopic dermatitis, familial psoriasis, and epidermodysplasia verruciformis), various heart disorders for example, myocardial necrosis, cardiomegaly, cardiac failure, myocardial infarction, ischemia, coronary artery disease, atherosclerosis or angina pectoris, or any other disorders (e.g., tumors, cancers, leukemia, myeloid diseases, and traumas) which are directly or indirectly associated with aberrant TREM-4 and/or TREM-5 activity and/or expression. The nucleic acids, polypeptides, and modulators thereof of the invention can be used to treat these disorders and diseases. Further, the nucleic acids, polypeptides, and modulators thereof of the invention can be co-administered with other therapeutics/prophylactics relevant Lo the diseases, e.g., anti-inflammatory agents, such as anti-TNF-α antibody, IL-1 receptor antagonist, anti-MIF antibody, and anti-HMG-I antibody, and chemotherapeutic agents.

The subjects to which the therapeutic and prophylactic and contraceptive methods of the present invention are applicable may be any mammalian or vertebrate species, which include, but are not limited to, cows, horses, sheep, pigs, fowl (e.g., chickens), goats, cats, dogs, hamsters, mice, rats, monkeys, rabbits, chimpanzees, and humans. In a preferred embodiment, the subject is a human.

### 5.8.2 Prophylactic Methods

In one aspect, the invention provides a method for preventing in a subject, a disease or condition associated with an aberrant expression or activity of a polypeptide of the invention, by administering to the subject an agent which modulates expression or at least one activity of the polypeptide. Subjects at risk for a disease which is caused or contributed to by aberrant expression or activity of a polypeptide of the invention can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The prophylactic agents described herein, for example, can be used to treat a subject at risk of developing disorders such as disorders discussed for example, in sections above relative to the uses of the sequences of the invention.

### 5.8.3 Therapeutic Methods

Another aspect of the invention pertains to methods of modulating expression or activity of a polypeptide of the invention for therapeutic purposes. The modulatory method of the invention involves contacting a cell with an agent that modulates one or more of the activities of the polypeptide, in other words “a modulator of TREM-4 actvity” or “a modulator of TREM-5 activity”. An agent that modulates activity can be an agent as described herein, such as a nucleic acid or a protein, a naturally-occurring cognate ligand of the polypeptide, a peptide, a peptidomimetic, or other small molecule. In one embodiment, the agent stimulates one or more of the biological activities of the polypeptide. Examples of such stimulatory agents include the active polypeptide of the invention and a nucleic acid molecule encoding the polypeptide of the invention that has been introduced into the cell. In another embodiment, the agent inhibits one or more of the biological activities of the polypeptide of the invention. Examples of such inhibitory agents include antisense nucleic acid molecules and antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a polypeptide of the invention. In one embodiment, the method involves administering an agent (e.g., an agent identified by a screening assay described herein), or combination of agents that modulates (e.g., upregulates or downregulates) expression or activity. In another embodiment, the method involves administering a polypeptide of the invention or a nucleic acid molecule of the invention as therapy to compensate for reduced or aberrant expression or activity of the polypeptide.

Stimulation of activity is desirable in situations in which activity or expression is abnormally low or down-regulated and/or in which increased activity is likely to have a beneficial effect. Conversely, inhibition of activity is desirable in situations in which activity or expression is abnormally high or upregulated and/or in which decreased activity is likely to have a beneficial effect.

In a specific embodiment, the modulator is a soluble form of TREM-4 (including TREM-4-alpha and TREM-4-beta) or TREM-5 molecule, for example, a fusion protein such as TREM-4-IgG1 or TREM-5-IgM as described in the previous sections. TREM-4 or TREM-5 may be used as a therapeutic agent when administered in an early phase of inflammation induced by LPS, presumably reducing the total amount of inflammatory mediators and preventing an irreversible tissue damages.

In another specific embodiment, an inhibitory antibody specific for TREM-4 (including TREM-4-alpha and TREM-4-beta) or TREM-5 molecule can be used as a therapeutic agent. Such antibodies would act as an antagonist against TREM-4 or TREM-5 by blocking the ligand-binding sites of TREM-4 and TREM-5 without triggering subsequent signal transduction reactions which lead to inflammatory disorders.

In another embodiment, nucleic acids comprising sequences encoding antibodies or fusion proteins, are administered to treat, prevent or ameliorate one or more symptoms associated with a disease, disorder, or infection, by way of gene therapy. Gene therapy refers to therapy performed by the administration to a subject of an expressed or expressible nucleic acid. In this embodiment of the invention, the nucleic acids produce their encoded antibody or fusion protein that mediates a therapeutic or prophylactic effect.

Any of the methods for gene therapy available in the art can be used according to the present invention. Exemplary methods are described below.

For general reviews of the methods of gene therapy, see Goldspiel et al., 1993, Clinical Pharmacy, 12:488-505; Wu and Wu, 1991, Biotherapy, 3:87-95; Tolstoshev, 1993, Ann. Rev. Pharmacol. Toxicol., 32:573-596; Mulligan, 1993, Science, 260:926-932); and Morgan and Anderson, 1993, Ann. Rev. Biochem., 62:191-217; May, 1993, TIBTECH, 11(5):155-215. Methods commonly known in the art of recombinant DNA technology which can be used are described in Ausubel et al., (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises nucleic acids encoding a polypeptide or an antibody of the invention, or fragments thereof, said nucleic acids being part of an expression vector that expresses the polypeptide or antibody of the invention in a suitable host. In particular, such nucleic acids have promoters, preferably heterologous promoters, operably linked to the coding region of the polypeptide or antibody of the invention, said promoter being inducible or constitutive, and, optionally, tissue-specific. In another particular embodiment, nucleic acid molecules of the invention are used in which the desired coding sequences are flanked by regions that promote homologous recombination at a desired site in the genome, thus providing for intrachromosomal expression of the polypeptide of the invention or fragments thereof (Koller and Smithies, 1989, Proc. Natl. Acad. Sci. USA, 86:8932-8935; and Zijlstra et al., 1989, Nature, 342:435438).

In another preferred aspect, a composition of the invention comprises nucleic acids encoding a fusion protein, said nucleic acids being a part of an expression vector that expresses the fusion protein in a suitable host. In particular, such nucleic acids have promoters, preferably heterologous promoters, operably linked to the coding region of a fusion protein, said promoter being inducible or constitutive, and optionally, tissue-specific. In another particular embodiment, nucleic acid molecules are used in which the coding sequence of the fusion protein and any other desired sequences are flanked by regions that promote homologous recombination at a desired site in the genome, thus providing for intrachromosomal expression of the fusion protein.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem., 262:44294432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188; WO 93/20221). Alternatively, the nucleic acid can be introduced intracellularly and incorporated within host cell DNA for expression, by homologous recombination (Koller and Smithies, 1989, Proc. Natl. Acad. Sci. USA, 86:8932-8935; and Zijlstra et al., 1989, Nature, 342:435-438).

In a specific embodiment, viral vectors that contain nucleic acid sequences encoding an antibody or a fusion protein are used. For example, a retroviral vector can be used (see Miller et al., 1993, Meth. Enzymol., 217:581-599). These retroviral vectors contain the components necessary for the correct packaging of the viral genome and integration into the host cell DNA. The nucleic acid sequences encoding the polypeptide of the invention, or fragments thereof, or a fusion protein to be used in gene therapy are cloned into one or more vectors, which facilitates delivery of the nucleotide sequence into a subject. Further details about retroviral vectors can be found in Boesen et al., 1994, Biotherapy, 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest., 93:644-651; Klein et al., 1994, Blood, 83:1467-1473; Salmons and Gunzberg, 1993, Human Gene Therapy, 4:129-141; and Grossman and Wilson, 1993, Curr. Opin. in Genetics and Devel., 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson (Current Opinion in Genetics and Development, 3:499-503, 1993), present a review of adenovirus-based gene therapy. Bout et al., (Human Gene Therapy, 5:3-10, 1994) demonstrated the use of adenovirus vectors to transfer genes to the respiratory epithelia of rhesus monkeys. Other instances of the use of adenoviruses in gene therapy can be found in Rosenfeld et al., 1991, Science, 252:431-434; Rosenfeld et al., 1992, Cell 68:143-155; Mastrangeli et al., 1993, J. Clin. Invest., 91:225-234; PCT Publication WO 94/12649; and Wang et al., 1995, Gene Therapy, 2:775-783. In a preferred embodiment, adenovirus vectors are used.

Adeno-associated virus (MV) has also been proposed for use in gene therapy (see, e.g., Walsh et al., 1993, Proc. Soc. Exp. Biol. Med., 204:289-300 and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol., 217:599-618; Cohen et al., 1993, Meth. Enzymol., 217:618-644; and Clin. Pharma. Ther., 29:69-92, 1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the subject.

In an embodiment in which recombinant cells are used in gene therapy, nucleic acid sequences encoding a polypeptide, an antibody or a fusion protein of the invention are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, 1992, Cell, 7 1:973-985; Rheinwald, 1980, Meth. Cell Bio., 21A:229; and Pittelkow and Scott, 1986, Mayo Clinic Proc., 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 5.9 Contraceptive Methods

The present invention provides methods of contraception by administration of modulators (i.e., agonists and antagonists) of TREM-4, including, but not limited to, TREM-4 polypeptides and variants, homologs, derivatives, and fragments thereof, the nucleic acids encoding the TREM-4 polypeptides, variants, homologs, derivatives, and fragments thereof, anti-TREM-4 antibodies, TREM-4 anti-sense molecules, etc. Since TREM-4 is associated with spermatogenesis, the methods of the invention are used to reduce or inhibit spermatogenesis by administering to a male subject in which contraception is desired a modulator of TREM-4. (Alternatively, modulators of TREM-4 that increase and promote spermatogenesis may be administered to treat infertility and/or improve fertility.)

In a particular embodiment, a TREM-4 polypeptide, a variant, homolog, derivative, or fragment thereof, or nucleic acid encoding the same, are administered as a vaccine to promote an immune response that inhibits or reduces spermatogenesis.

The contraceptive methods of the invention may be used in conjunction with another contraceptive method, such as, but not limited to, barrier methods such as use of condoms or diaphragms or cervical caps, or intravaginal use of contraception compounds such as non-oxynol-9, intrauterine devices, birth control pills or implants, etc.

### 6. EXAMPLES

The following examples illustrate the cloning, production, isolation, and characterization of TREMs and fusion proteins thereof, and antibodies. These examples should not be construed as limiting.

### 6.1 TREM-4

### 6.1.1 Cloning of TREM-4 cDNAs

GenBank expressed sequence tagged database (dbEST) was searched with the amino acid sequences of TREM-1, -2, -3, CMRF-35 and CMRF-35H using the tblastn algorithm. One sequence (accession no. U70073) with no matches in the GenBank non-redundant database (nr) was selected. This sequence contained an open reading frame encoding the leader sequence and the extracellular domain of TREM-4 putative polypeptide. The TREM-4 cDNA fragment encoding the transmembrane and cytoplasmic domain of TREM-4 was cloned by Rapid Amplification of cDNA ends (RACE) (see below).

### 6.1.2 RNA Blot Assay

RNA analysis was performed on human normal tissues RNA blots (MTN blot I and II, Clontech, Palo Alto, Calif.). Membranes were hybridized with TREM-4 or β-actin (Clontech) cDNA probes labeled with [32P]dCTP. TREM-4 cDNA probe spanned nucleotides 61 to 420 and was amplified from human bone marrow RNA (Clontech) by reverse transcriptase-polymerase chain reaction (RT-PCR) using the following oligonucleotides: 5′-AATGCGGCTTCTGGTCCTGCT-3′ (SEQ ID NO:47) 5′-ATCAGTAAAGACTCATCGGGG-3′ (SEQ ID NO:48). Hybridization, washings and exposures were carried out by standard techniques.

### 6.1.3 Race

Total RNA derived from testis and bone marrow cells (Clontech) was used for the preparation of first-strand cDNA by reverse transcriptase using the following oligo(dT)-adapter: 5′-ACTCGAGTCGACATCGATTTTTTTTTTTTTTTTT-3′ (SEQ ID NO:49). The 3′ end of TREM-4 was amplified from oligo(dT)-adapter-primed cDNA by two rounds of polymerase chain reaction (PCR) using two 5′ nested primers and a 3′ adapter primer. 5′ nested primers were: 5′-AGACGCTGGGGAGTACTGGT-3′ (SEQ ID NO:50) and 5′-GTGGGTCGAGAAACGGG-3′ (SEQ ID NO:51). The 3′ adapter primer was: 5′-GACTCGAGTCGACATCG-3′ (SEQ ID NO: 52). cDNA synthesis and PCR were performed following a standard protocol. The amplified cDNA fragments were separated by electrophoresis in a 1.5% agarose gel, purified by Qiaex II (Qiagen, Hilden, Germany), cloned into pCR2.1 (Invitrogen, Carlsbad, Calif.) and sequenced with the following oligonucleotides: T7 (Invitrogen), M13 reverse (Invitrogen) 5′-CCCAGGGTGTCCATCCCGAT-3′ (SEQ ID NO:53) and 5′-CCTTGCTCCACAGGAGCAGG-3′ (SEQ ID NO:54).

### 6.1.4 Alignments

Human Ig-like V-type domains were aligned by the Clustal method using the ClustalW function of MacVector (Oxford Molecular Group, pic). Accession numbers for the molecules used in the alignment are: TREM-4 (AF427619/AF427620), TREM-5 (AF427618), NKp44 (SEQ ID NO:11), CMRF35 (SEQ ID NO:9), polymeric immunoglobulin receptor (PIGR) (SEQ ID NO:10), TREM-1 (SEQ ID NO:13), TREM-3 (SEQ ID NO:14) and TREM-2 (SEQ ID NO:12).

### 6.1.5 Chromosomal Localization

To identify the chromosomal localization of TREM-4, GenBank Human Genome Sequence was searched with the nucleotide sequence of TREM-4 using the “blastn” algorithm, as known in the art.

### 6.1.6 Production of TREM-4 Human IgG1 (TREM-4-HulgG) Fusion Protein

To produce TREM-4 as a soluble fusion protein, the inventors constructed a chimeric gene consisting of the TREM-4 extracellular domain and human IgG1 constant regions. The cDNA fragment encoding the leader sequence and extracellular region of TREM-4 was amplified by PCR from cloned plasmid DNA. The forward primer (TAGTAGGAATTCCCCAGAATGCGGCTTCTGGTCCTGCTATG, SEQ ID NO:55) contained an EcoRI restriction site and the TREM-4 start codon. The reverse primer (TAGTAGAAGCTTATACTTACCCCTGGGCTTAGAGCTGCCAC, SEQ ID NO:56) provided a HindIII restriction site, a splice donor sequence, and several TREM-4 codons several TREM-4 codons preceding the transmembrane domain. The PCR product was cut with EcoRI and HindIII, and ligated into an expression vector containing the exons for hinge, CH2 and CH3 region of human IgG1, the guanosine phosphotransferase gene conferring resistance to mycophenolic acid, and the k promoter for the expression in the mouse myeloma cell line J558L. The expression vector used was based on the plasmid pCD4 (Traunecker et al., 1991, Trends Biotechnol., 9:109) which is derived from plasmid pHT4-Y1 which can be prepared as previously described by Traunecker, Luke and Karjalainen in Nature 331, 84-86 (1988) and EP0394827. Transfection of the chimeric gene into the mouse myeloma cell line J558L, screening of culture supernatants, and purification of huTREM-I-IgG1 were performed as previously described (Traunecker, et al., 1991, Trends Biotechnol. 9:109). Briefly, the huTREM-4-IgG1 plasmid was transfected into J558L mouse myeloma cells by electroporation and cells were cultured in DMEM supplemented with 2 mM L-glutamine, 1% non-essential amino acids, 1% sodium pyruvate, 50 μg/ml kanamycin. After two days of culture, selective medium containing 4 μg/ml mycophenolic acid (Calbiochem) and 125 μg/ml xanthine (Sigma) was added and incubation at 37° C. continued until resistant colonies appeared. Clones were screened for production of soluble IgG fusion proteins by enzyme-linked immunosorbent assay (ELISA) using a goat anti-human IgG antibody. Producer clones were expanded, while the FCS content was diminished to 2%. For purification of the fusion protein, culture supernatant was concentrated and adsorbed over a recombinant protein A column (Repligen, Cambridge, Mass.). After washing with PBS-0.02% sodium azide, the bound fusion protein was eluted with 0.1M glycine-HCl, pH 2.65. One (1)-ml fractions were collected in test tubes containing 100 μl 2MTris-HCl, pH 8, pooled, and dialyzed against PBS. Purified protein was then concentrated, sterile-filtered and kept frozen.

### 6.1.7 Production of Anti-TREM-4 Monoclonal Antibodies (mAb)

BALB/c mice received an initial subcutaneous injection of 200 μg purified TREM-4-HulgG1 behind the neck. Two weeks later they were given a booster immunization of 10 μg purified TREM-4-HulgG1 in PBS intravenously. Three days later spleen cells were isolated and fused with the Sp2/0 myeloma cells. Hybridoma supernatants were screened in two steps. First, an ELISA was performed using TREM-4-HuIgG1 in the coating step and human-adsorbed alkaline phosphatase-labeled goat-anti-mouse IgG (PharMingen, San Diego, Calif.) as detecting antibody. Supernatants from clones that were positive in ELISA were then tested by staining of 293 cells transiently expressing TREM-4 cDNA in flow cytometry (see below). Three clones termed TR4.1, TR4.2 and TR4.3 were obtained.

### 6.1.8 Transfections

A TREM-4 cDNA fragment encoding the extracellular region of TREM-4 was amplified by PCR from cloned plasmid DNA. The forward primer (TAGTAGAGATCTTATGAAGCCCTGGAGGGGCCAGA, SEQ ID NO:57) contained a BglII restriction site. The reverse primer (TAGTAGGTCGACGGACACCCTGGGCTTAGAGCT, SEQ ID NO:58) provided a SalI restriction site. The PCR product was cut with BglII and SalI, and ligated into the expression vector pDisplay (Invitrogen). The cDNA fragment was in frame with the vector's N-terminal secretion signal and C-terminal myc epitope and transmembrane anchoring domain of platelet-derived growth factor receptor (PDGFR). TREM-4/pDisplay was transiently expressed in 293 cells using lipofectin (Bethesda Research Laboratories, Gaithersburg, Md.). Expression of TREM-4/pDisplay was confirmed by flow cytometry using an anti-myc antibody (Invitrogen).

### 6.1.9 Immunohistochemistry

Human tissue samples included lymph nodes, skin, thymus, liver and testis. All tissues were fresh frozen in isopentane that had been previously cooled in liquid nitrogen and stored at −80° C. Immunostaining was performed on frozen sections, applying the anti-TREM-4 antibody at the concentration of ˜1 μg/ml, followed by the Labelled Streptavidin-Biotin (LSAB) procedure (Dako, Denmark). Nuclei were counterstained with Mayer's hematoxylin.

### 6.1.10 Human Multiple Tissue Expression Array

TREM-4 gene expression was profiled on a MTE™ array (BD Biosciences) consisting of 75 tissue-specific poly A⁺ RNAs directly spotted on a nylon membrane. The hybridization was performed according to the procedure provided by the manufacturer. The probe was prepared with Random Prime DNA Labelling Mix (-dCTP) (Sigma) and radiolabelled with ³²P.

### 6.1.11 Relative Quantitation of TREM-4 mRNA with Real Time PCR

Total mRNA was purified with Quiagen RNeasy kit with DNasel digestion step. cDNA was transcribed from a total of 1 μg purified RNA with the TaqMan® Reverse Transcription kit (Applied Biosystems). 10 ng of cDNA was used to amplify TREM-4 transcripts with specific primers (TREM-4 forward primer: TGC TCT GGC CAC ATC TAT GCA, SEQ ID NO: 66; TREM-4 reverse primer: TTT CTC GAC CCC ACA CCA GTA, SEQ ID NO: 67) and β-actin was used as an endogenous control (β-actin forward primer: GAC GGG GTC ACC CAC ACT GTG CCC ATC TA, SEQ ID NO: 68; β-actin reverse primer: CTA GAA GCA TTT GCG GTG GAC GAT GGA GGG, SEQ ID NO: 69) for normalization of RNA quantity. Real time PCR was performed on a ABI PRISM® 7000 Sequence Detection System (Applied Biosystems). Cycle-by-cycle detection of accumulated PCR product was made possible by using SYBR® Green as fluorescent dye. Standard curves (ranging from 10 ng to 0.001 ng of cDNA) were prepared for both TREM-4 and β-actin using cDNA from a positive control sample (TREM-4 transfected cells). The standard curve correlates the amount of cDNA present in the sample (log of RNA concentration) with the cycle threshold (Cₜ). Each cDNA sample was analyzed in triplicate and mean value of Cₜ was considered. For each sample, the amount of TREM-4 or β-actin mRNA was determined from the corresponding standard curve. The calculated quantity of TREM-4 mRNA was then divided by the calculated quantity of β-actin mRNA to obtain a normalized value of TREM-4.

### 6.1.12 Immunoprecipitation with Anti Human TREM-4 Monoclonal Antibodies

Cells or tissues were homogenized in lysis buffer (10 mM Tris pH7.4, 150 mM NaCl, 1 mM EDTA pH8.0, 1% Triton X-100, 10% glycerol, complete protease inhibitors (Roche)). Lysates were clarify by high speed centrifugation and total protein content measured by Bradford assay. For each sample 500 μg of total protein extract was used to immunoprecipitate TREM-4 with two different monoclonal mouse anti-human TREM-4 antibodies (64F5 and 64A9) or with the same quantity of a non-relevant isotype matched antibody as a negative control. Immune-complexes were then purified with Protein G-Sepharose and loaded on a reducing SDS-PAGE. Proteins were blotted on nylon membrane and western blot was performed with biotinilated mouse anti-human TREM-4 (clone 64F5) revealed with an anti-biotin monoclonal antibody.

### 6.1.13 Staining and Cytofluorimetric Analysis

Primary cells and cell lines were stained with huTREM-4/IgG. Briefly, cells were pre-incubated with human IgG (Sigma) to block free Fc binding sites. Supernatant (100 μl=2 μg) of soluble TREM-4/IgG was added and incubated 30 minutes at 4° C. After washing, 1 μl of F(ab)₂ donkey anti-human IgG1-PE (Jackson Immunoresearch) were added. After washing, cells were resuspended and analyzed by flow cytometry (FACS LSR, Becton Dickinson). Negative control was performed staining cells with purified human IgG.

For intracellular staining with huTREM-4/IgG fusion protein, cells were fixed with 2% paraformaldehyde/PBS and permeabilized with 0.5% saponin/PBS. Non-specific binding to Fc receptors were prevented by blocking with 50 μg of human IgG. Cells were stained with 1 μg of huTREM-4/IgG fusion protein and revealed with a PE-conjugated donkey anti-human IgG antibody.

### 6.2 TREM-5

### 6.2.1 Cloning of TREM-5 cDNA

GenBank Human Genome Sequence was searched with the amino acid sequences of TREM-1 (SEQ ID NO:13), TREM-2 (SEQ ID NO:12), TREM-3 (SEQ ID NO:14), CMRF-35 (SEQ ID NO:9) and CMRF-35H using the tblastn algorithm. A genomic DNA sequence encoding TREM-5 Ig-SF domain was found (NT_{—}010672.8|Hs17_{—}10829 Homo sapiens chromosome 17 working draft sequence segment, nucleotides 495348-495019). The 3′ and 5′ ends of TREM-5 cDNA fragment were cloned by from bone marrow RNA by Rapid Amplification of cDNA ends (RACE) using the Marathon™ cDNA Amplification Kit (Clontech). TREM-5 full length cDNA was amplified from human bone marrow RNA (Clontech) by reverse transcriptase-polymerase chain reaction (RT-PCR). The inventors prepared first-strand cDNA from bone marrow cell RNA (Clontech) by reverse transcriptase using the following oligo(dT)-adapter: 5′-ACTCGAGTCGACATCGATTTTTTTTTTTTTTTTT-3′ (SEQ ID NO:59). TREM-5 was amplified from oligo(dT)-adapter-primed cDNA by PCR using with the following primers: 5′-ACGAGGAGCCGGGAAGGCAGA-3′ (SEQ ID NO:60) and 5′-AGGCTCTGCAGATCCATCTC-3′ (SEQ ID NO:61). cDNA synthesis and PCR were performed following a standard protocol. The amplified cDNA fragments were separated by electrophoresis in a 1.5% agarose gel, purified by Qiaex II (Qiagen, Hilden, Germany), cloned into pCR2.1 (Invitrogen, Carlsbad, Calif.) and sequenced with the T7 and M13 reverse oligonucleotides (Invitrogen).

### 6.2.2 Alignments

Human Ig-like V-type domains were aligned by the Clustal method using the ClustalW function of MacVector (Oxford Molecular Group, plc). Accession numbers for the molecules used in the alignment are: TREM-4 (AF427619/AF427620), TREM-5 (AF427618), NKp44 (SEQ ID NO:11), CMRF35 (SEQ ID NO:9), polymeric immunoglobulin receptor (PIGR) (SEQ ID NO:10), TREM-1 (SEQ ID NO:13), TREM-3 (SEQ ID NO:14) and TREM-2 (SEQ ID NO:12).

### 6.2.3 Chromosomal Localization

To identify the chromosomal localization of TREM-5, GenBank Human Genome Sequence was searched with the nucleotide sequence of TREM-5 using the blastn algorithm.

### 6.2.4 Production of TREM-5 Human IgG1 (TREM-5-HuIgG) Fusion Protein

To produce TREM-5 as a soluble fusion protein, the inventors constructed a chimeric gene consisting of TREM-5 extracellular domain and human IgG1 constant regions. The cDNA fragment encoding the leader sequence and extracellular region of TREM-5 was amplified by PCR from cloned plasmid DNA. The forward primer (TAGATGGAATTCATGTGGCTGCCCCCTGCTCTGCT, SEQ ID NO:62) contained an EcoRI restriction site and the TREM-5 start codon. The reverse primer (TAGTAGAAGCTTATACTTACCGTAGTGGTTCCTCTTGTGGGAG, SEQ ID NO:63) provided a HindIII restriction site, a splice donor sequence, and several TREM-5 codons preceding the transmembrane domain. The PCR product was cut with EcoRI and HindIII, and ligated into an expression vector containing the exons for hinge, CH2 and CH3 region of human IgG1, the guanosine phosphotransferase gene conferring resistance to mycophenolic acid, and the k promoter for the expression in the mouse myeloma cell line J558L. Transfection, screening of culture supernatants and purification of TREM-5 human IgG1 (TREM-5-HuIgG) were performed as described herein for TREM-4-HuIgG.

### 6.2.5 Production of anti-TREM-5 Monoclonal Antibodies (mAb)

BALB/c mice were immunized with purified TREM-5-HuIgG1 behind the neck. Two weeks later they were given a booster immunization of 10 μg purified TREM-5-HuIgG1. Spleen cells were isolated and fused with the Sp2/0 myeloma cells. Hybridoma supernatants were screened in two steps. First, an ELISA was performed using TREM-5-HuIgG1 in the coating step and human-adsorbed alkaline phosphatase-labeled goat-anti-mouse IgG (PharMingen, San Diego, Calif.) as detecting antibody. Supernatants from clones that were positive in ELISA were then tested by staining of 293 cells transiently expressing TREM-5 cDNA in flow cytometry (see below). The inventors obtained two hybridoma clones termed TR5.1 and TR5.2.

### 6.2.6 Transfections

A TREM-5 cDNA fragment encoding the extracellular, transmembrane and cytoplasmic regions of TREM-5 was amplified by PCR from cloned plasmid DNA. The forward primer (TAGTAGAAGCTTGAGTCTGTGAGAGCCCCAGAGCAGGGG, SEQ ID NO:64) contained a HindIII restriction site. The reverse primer (TAGTAGTCTAGACTCTGCAGATCCATCTCTCTAAGT, SEQ ID NO:65) provided a XbaI restriction site. The PCR product was cut with HindIII and XbaI, and ligated into the expression vector pCMV1FLAG (Sigma). The cDNA fragment was in frame with the vector's N-terminal secretion signal and FLAG epitope. TREM-5/pCMV1FLAG was transiently expressed in 293 cells using lipofectin (Bethesda Research Laboratories, Gaithersburg, Md.). Expression of TREM-5/pCMV1FLAG was confirmed by flow cytometry using an anti-FLAG antibody (Sigma).

### 6.2.7 Human Multiple Tissue Expression Array

TREM-5 gene expression was profiled on a MTE™ array (BD Biosciences) consisting of 75 tissue-specific poly A⁺ RNAs directly spotted on a nylon membrane. The hybridization was performed according to the procedure provided by the manufacturer. The probe was prepared with Random Prime DNA Labelling Mix (-dCTP) (Sigma) and radiolabelled with ³²P.

### 6.2.8 Relative Quantitation of TREM-5 mRNA with Real Time PCR

Total mRNA was purified with Quiagen RNeasy kit with DNasel digestion step. cDNA was transcribed from a total of 1 μg purified RNA with the TaqMan® Reverse Transcription kit (Applied Biosystems). 10 ng of cDNA was used to amplify TREM-5 transcripts with specific primers (TREM-5 forward primer: CCC CTG CTC TGC TCC TTC TC, SEQ ID NO: 70; TREM-5 reverse primer: CAC CCC TCG GCA CCA CCA C, SEQ ID NO: 71) and β-actin was used as an endogenous control (β-actin forward primer: GAC GGG GTC ACC CAC ACT GTG CCC ATC TA, SEQ ID NO: 72; β-actin reverse primer: CTA GM GCA TTT GCG GTG GAC GAT GGA GGG, SEQ ID NO: 73) for normalization of RNA quantity. Real time PCR was performed on a ABI PRISM® 7000 Sequence Detection System (Applied Biosystems). Cycle-by-cycle detection of accumulated PCR product was made possible by using SYBR® Green as fluorescent dye. Standard curves (ranging from 10 ng to 0.001 ng of cDNA) were prepared for both TREM-5 and β-actin using cDNA from a positive control sample (TREM-5 transfected cells). The standard curve correlates the amount of cDNA present in the sample (log of RNA concentration) with the cycle threshold (Cₜ). Each cDNA sample was analyzed in triplicate and mean value of Cₜ is considered. For each sample, the amount of TREM-5 or β-actin mRNA was determined from the corresponding standard curve. The calculated quantity of TREM-5 mRNA is then divided by the calculated quantity of β-actin mRNA to obtain a normalized value of TREM-5.

### 6.2.9 Purification of Human Dendritic Cells

PBMCs were isolated from buffy coats by Ficoll gradient (Pharmacia Biotec AB, Uppsala, Sweden) and monocytes were purified from peripheral bloo with MACS Monocyte Isolation Kit (Miltenyi Biotec, Bergish Gladblach, Germany) via negative selection of CD14⁺ cells. The isolated CD14⁺ cells obtained were cultured in RPMI+5% FC, GM-CSF 800 U/ml (Mielogen 300, Schering-Plough S.p.A.), IL-4 1000 U/ml (PharMingen). Supplements were freshly added every other day. At day 6, dendritic cells dfferention was checked by staining with anti-CD1a, anti-mannose receptor, anti-CD83 (R&D System). Maturation of DC was induced with SAC (Calbiochem) 0.1% or with an anti-CD40 Mab coated on a plate (10 μg/ml) for 36 hrs and maturation is evaluated by staining with anti-CD83, anti-CD86 and anti-mannose receptor MAbs (Pharmingen).

Peripheral blood myeloid dendritic cells (M-DCs) and dendritic cells (P-DCs) were magnetically sorted with BDCA-1 and BDCA-4 cell isolation kits (Miltenyi Biotec, Bergish Gladblach, Germany), respectively, as described (Dzionek, A., et al, 2000 J. Immunol. 165:6037), to a purity of 95-98% in both cases.

### 6.3 Results

### 6.3.1 TREM-4 is a Novel Transmembrane Protein of Ig-SF

Using the amino acid sequences of TREM-1, -2, -3 and CMRF-35 polypeptides, one cDNA encoding the leader sequence and the extracellular region of a novel member of the immunoglobulin superfamily was identified and named TREM-4. The TREM-4 cDNA sequence was completed by RACE, yielding two TREM-4 isoforms with distinct 3′ ends, called TREM-4-alpha (accession no. AF427619; SEQ ID NO:1 of FIG. 1) and TREM-4-beta (accession no. AF427620; SEQ ID NO:2 of FIG. 2). Both amino acid sequences have identical hydrophobic signal peptide, extracellular region composed of a single Ig-SF domain and transmembrane domain containing a charged arginine residue. The length of the Ig-fold and the characteristic pattern Asp-x-Gly-x-Tyr-x-Cys in the region leading to the β-strand F indicate that the Ig-fold is of the V-type (SEQ ID NO:18 of FIG. 4; SEQ ID NO:24 of FIG. 5). The Ig-SF domain contains one potential N-glycosylation site. TREM-4-alpha continues with a cytoplasmic tail of 50 amino acids with no signaling motifs (SEQ ID NO:20 of FIG. 4); TREM-4-beta shows a cytoplasmic tail of 39 amino acids (SEQ ID NO:26 of FIG. 5). Most likely, TREM-4-alpha and -beta represent alternatively spliced forms of the same transcript. TREM-4 transmembrane domain is similar to those present in activating NK cell and myeloid cell receptors that pair with the transmembrane adapter proteins DAP12/KARAP, CD3ζ or FcRγ. Thus, it is likely that TREM-4 is associated with one of these adapters.

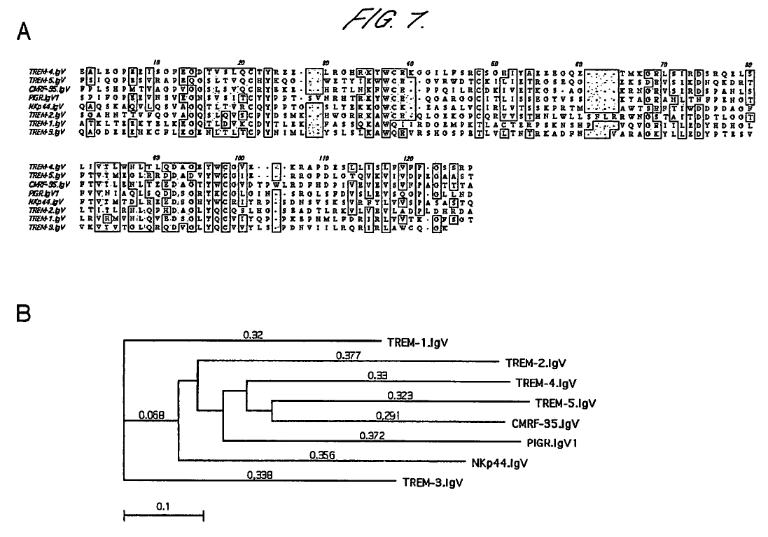

Comparison of TREM-4 extracellular Ig-SF domain with protein sequence databases revealed closest similarity with TREM-5 and CMRF-35 and a more distant similarity with PIGR, NKp44 and TREM-1, -2 and -3 (FIGS. 7A-B). Alignment of all these Ig-SF domains showed conservation of 2 cysteine residues that are likely to generate the typical intrachain disulfide bonds of Ig-SF folds. In addition, TREM-4 displayed 2 more cysteines at amino acids 51 and 61, which may form a second intrachain bond.

### 6.3.1.1 TREM-4 Maps on Human Chromosome 17

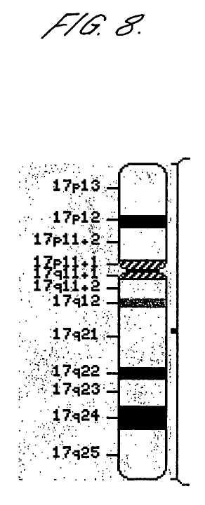

The GenBank human genome sequence data base was searched with TREM-4 polypeptide using the blast algorithm. TREM-4 locus was identified on human chromosome 17q21 within the homo sapiens chromosome 17 working draft sequence segment (NT_{—}010755.8H|s17_{—}10912, Length=1172625) (FIG. 8).

### 6.3.1.2 Expression of TREM-4

RNA blot analysis of multiple human tissues with a TREM-4 cDNA shows a

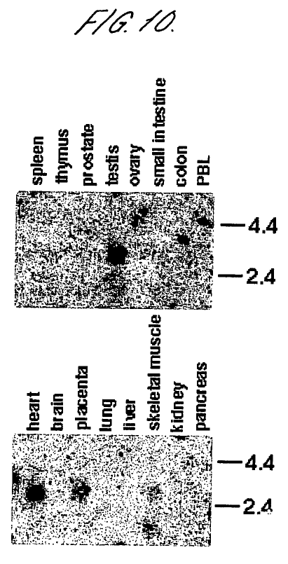

transcript of ˜2.4 kb in the testis, heart, placenta and skeletal muscle (FIG. 10). Immunohistochemical analysis shows that TREM-4 is selectively expressed in the endothelium of capillaries and in the testis. TREM-4 is strongly expressed in the capillaries of subcutaneous adipose tissue (FIG. 11A), lymph nodes (FIG. 11B), thymus (FIGS. 11C-D), as well as in liver sinusoid endothelium cells (FIG. 11E). However, capillaries of dermis, lung and placenta lack expression of TREM-4. Endothelium of arteries, arterioles, veins, venules and lymphatic vessels are TREM-4 negative. High endothelium venules are negative as well. Inflammation or neoplastic transformation are associated with reduced or lack of TREM-4-positive endothelial cells. In the testis, TREM-4 expression is detectable in the spermatogenic cells of seminiferous tubules (FIGS. 12A-D), whereas Sertoli cells and Leydig cells are negative (FIGS. 13A-B).

### 6.3.1.3 Function of TREM-4

The selective expression of TREM-4 in the capillaries and testis indicates that TREM-4 may have an important role in the microcirculation and male fertility. Capillaries and testis contain high levels of proteoglycans (PG). PG consist of a core protein and an associated glycosaminoglycan (GAG) chain of heparan sulfate, chondroitin sulfate, dermatan sulfate or keratan sulfate, which are attached to a serine residue. The core proteins of PG may be transmembrane proteins, e.g., syndecan, GPI-anchored proteins, e.g., glypican, or extracellular matrix (ECM) proteins, e.g. testican. Thus, it is possible to hypothesize that TREM-4 is a receptor for PG, allowing interaction of endothelial cells and germ cells with different cell surface and matrix proteoglycan core proteins.

In the capillaries, interaction of TREM-4 with PG may be responsible for the binding of endothelial cells to ECM proteins, contributing to normal morphogenesis and function of capillaries. Lack of this interaction may compromise a normal function of capillaries.

In the testis, the ECM plays a fundamental role in testicular development, morphogenesis, and spermatogenesis. Specifically, the ECMs of the testicular lamina propria mediates cell-cell interactions between Sertoli, myoid, Leydig and germ cells. Thus, binding of germ cells to testicular ECM through TREM-4 may regulate spermatogenesis, adherence of germ cells to Sertoli cells and testicular cord formation.

### 6.3.1.4 Expression of TREM-4

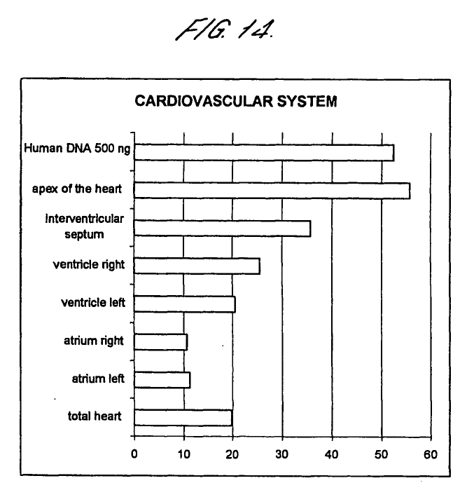

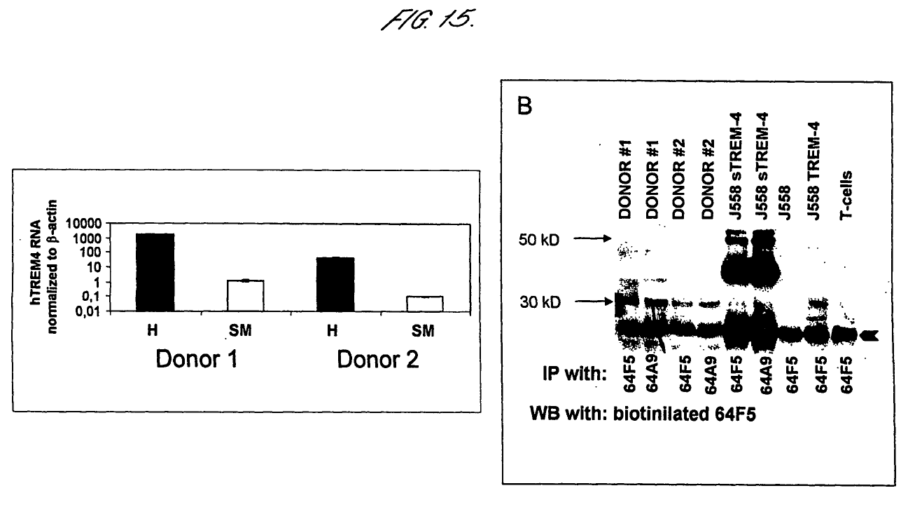

In order to determine where TREM-4 is most abundantly expressed, the inventors analyzed a pre-made MTE Array consisting of 75 tissue-specific poly A+ RNAs directly spotted on a nylon membrane. An extensive analysis of a wide variety of foetal and adult tissues, showed that TREM-4 mRNA levels are relatively abundant in heart tissues and also detected, although at lower levels, in skeletal muscle and in the central nervous system (FIG. 14). TREM-4 mRNA is not detectable in tissues of foetal origin, hematopoietic origin, in the digestive system, and in the urinary system. Quantitative PCR performed on autoptic heart-trauma specimens from three different donors indicate that TREM-4 mRNA is specifically expressed in heart and at lower levels in skeletal muscle of the same donors (FIG. 15, panel A). Immunoprecipitation of TREM-4 protein from heart tissues with two different specific anti-TREM-4 monoclonal antibodies (64A9 and 64F5) and subsequent western blot analysis with a biotinylated anti TREM-4 monoclonal antibody (64F5.bio) revealed a band at 30 kD in heart tissues from two different donors. Immunoprecipitation of TREM-4 from TREM-4 transfected cells revealed a band of 30 kD correspondent to the expected size of protein expressed, that is not detected in non-transfected cells (FIG. 15, panel B). Immunoprecipitation from supernatant of J558 cells expressing a soluble fusion protein huTREM-4/IgG revealed a band of approximately 50 kD corresponding to the expected molecular weight of the fusion protein. Altogether these data demonstrate that TREM-4 is expressed in the heart.

### 6.3.1.5 Expression of TREM-4 Ligand

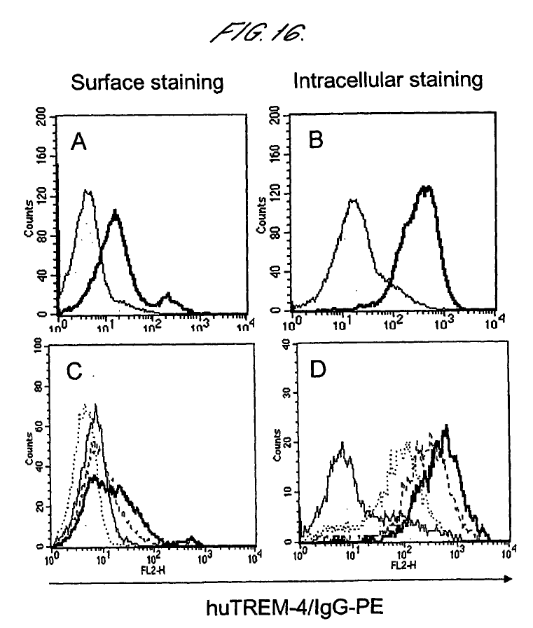

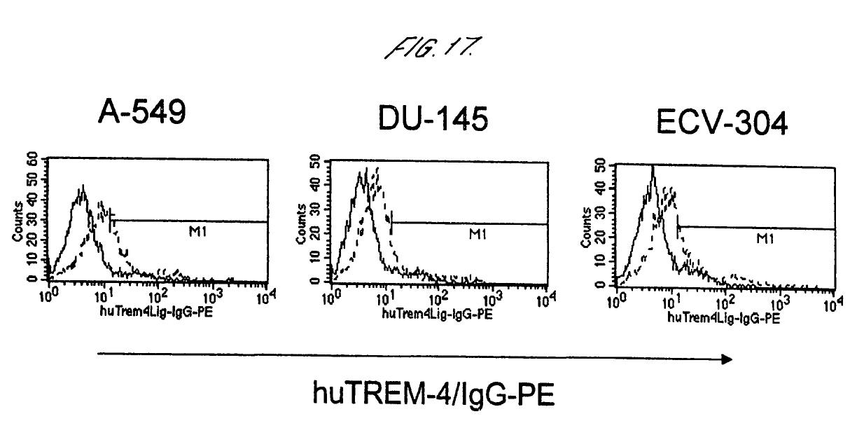

In order to characterize the expression of TREM-4 ligand, the inventors have analyzed a large panel of cell lines and primary cells obtained from different human tissues. Staining was performed as described above with huTREM-4/IgG. TREM-4 ligand expression was specifically detected on epithelial cells lines and on primary epithelial cells. FIG. 16 shows that TREM-4 ligand is expressed both at the cell surface and intra-cellularly (panels A and B). The staining with huTREM-4/gG was specific and dose dependent. As shown in FIG. 16 (panels C and D), when A549 cell were stained with different amounts of huTREM-4/IgG the number of positive cells was proportional to the amount of huTREM-4/IgG. Expression of TREM-4 ligand is also detected on cells of the prostate carcinoma cell lines DU-145 and ECV-304 and on primary bronchial epithelial cells (HBE) (FIG. 17 and data not shown).

### 6.3.2 TREM-5 is a Novel Transmembrane Protein of Ig-SF

By searching the GenBank human genome sequence data base with TREM-1, TREM-2, TREM-3 and CMRF-35 polypeptides, a sequence encoding a novel immunoglobulin superfamily (Ig-SF) domain was identified. A cDNA sequence corresponding to such Ig-SF domain was amplified by RT-PCR from human bone marrow RNA. The 5′ and 3′ ends of this cDNA were subsequently obtained by RACE. The full length cDNA was amplified by RT-PCR from bone marrow RNA, cloned and sequenced. This molecule was named TREM-5 (accession n. AF427618; SEQ ID NO:3 of FIG. 3). TREM-5 begins with an hydrophobic signal peptide followed by ar, extracellular region composed of a single Ig-SF domain. The length of the Ig-fold and the characteristic pattern Asp-x-Asp-x-Tyr-x-Cys in the region leading to the β-strand F indicate that the Ig-fold is of the V-type (SEQ ID NO:29 of FIG. 6). The Ig-SF domain contains no potential N-glycosylation sites. TREM-5 continues with a transmembrane domain containing a charged lysine residue and a cytoplasmic tail of 29 amino acids. The lysine in the transmembrane region is reminiscent of that of activating NK cell and myeloid cell receptors that pair with the transmembrane adapter proteins DAP12. Thus, it is likely that TREM-5 is associated with DAP12. The cytoplasmic domains contains a tyrosine based motif YMNF. This short amino acid sequence contains the Y×N motif that has been previously shown to recruit the signaling protein Grb-2 (Chang C., Dietrich J., Harpur A. G., Lindquist J. A., Haude A., Loke Y. W., King A., Colonna M., Trowsdale J., and Wilson M. J., 1999, Cutting edge: KAP10, a novel transmembrane adapter protein genetically linked to DAP12 but with unique signaling properties. J. Immunol. 163:4651-54). Thus, TREM-5 may recruit Grb-2.

Comparison of TREM-5 extracellular Ig-SF domain with protein sequence databases revealed closest similarity with CMRF-35 and TREM-4 and a more distant similarity with PIGR, NKp44 and TREM-1, -2 and -3 (FIGS. 7A-B). Alignment of all these Ig-SF domains showed conservation of 2 cysteine residues that are likely to generate the typical intrachain disulfide bonds of Ig-SF folds. In addition, TREM-5 displayed 2 more cysteines at amino acids 50 and 58, which may form a second intrachain bond.

### 6.3.2.1 TREM-5 Maps on Human Chromosome 17

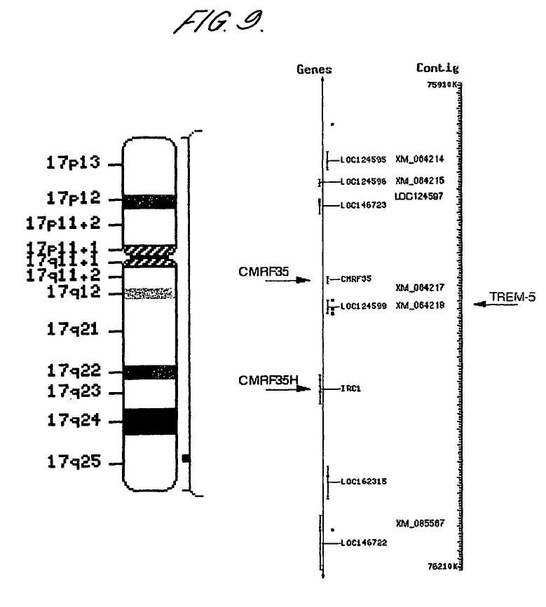

The GenBank human genome sequence data base was searched with TREM-5 polypeptide using the blast algorithm. TREM-5 locus was identified on human chromosome 17q25 within the homo sapiens chromosome 17 working draft sequence segment (NT_{—}010672.8|Hs17_{—}10829 Homo sapiens chromosome 17 working draft sequence segment, Length=939480) (FIGS. 9A-B).

TREM-5 is closely linked to the loci enccoding CMRF-35 and CMRF-35H (also called IRC1). In addition, this region contains 5 additional genes similar to TREM-5 and CMRF-35. At least two of these genes encode Ig-SF members with a residue of lysine in the transmembrane domain that may bind DAP12. Thus, TREM-5 is part of a novel gene cluster of related immunoreceptors on human chromosome 17q25. Interestingly, this region has been shown to contain susceptibility loci for three different skin diseases. Thus, TREM-5 may be implicated in these diseases (see below).

### 6.3.2.2 Expression of TREM-5

No expression of TREM-5 by norther blot analysis of multiple normal tissue blots and staining of peripheral blood leukocytes was detected. However, TREM-5 cDNA was amplified from bone marrow RNA. This indicates that TREM-may be expressed at low levels in a bone marrow-derived population of leukocytes and that TREM-5 may be upregulated in certain conditions, such as cell activation or inflammation.

### 6.3.2.3 Function of TREM-5

TREM-5 is located in a chromosomal region which has been associated with three skin diseases. Specifically, 17q25 contains a susceptibility locus for atopic dermatitis (Cookson et al., 2001, Genetic linkage of childhood atopic dermatitis to psoriasis susceptibility loci. Nature Genet. 27:372-73). In addition, 17q25 region was recently found to contain a dominant locus for the susceptibility to familial psoriasis (Bowcock et al., 2001, Insights into psoriasis and other inflammatory diseases from large-scale gene expression studies. Hum Mol Genet. 10:1793-805). Finally, susceptibility to epidermodysplasia verruciformis (EV) was found to map within the same chromosomal region (Ramoz et al., 2000, Evidence for a nonallelic heterogeneity of epidermodysplasia verruciformis with two susceptibility loci mapped to chromosome regions 2p21-p24 and 17q25. J. Invest. Derm. 114:1148-53). EV is a rare genodermatosis characterized by an abnormal predisposition to infection with the oncogenic human papillomavirus type (HPV5). Thus, these genetic studies indicate the presence of genes in the 17q25 region that modulate dermal responses to allergens, viruses and other pathogenic stimuli. TREM-5 is also encoded is in 17q25, it is probably expressed on a population of leukocytes and most likely mediates cell activation through association with DAP12. Thus, TREM-5 may be involved in the pathogenesis of these skin diseases by amplyfing dermal responses to pathogens. Blockade of TREM-5 with monoclonal antibodies or soluble TREM-5-HuIgG fusion protein may reduce or block skin diseases.

### 6.3.2.4 Expression of TREM-5

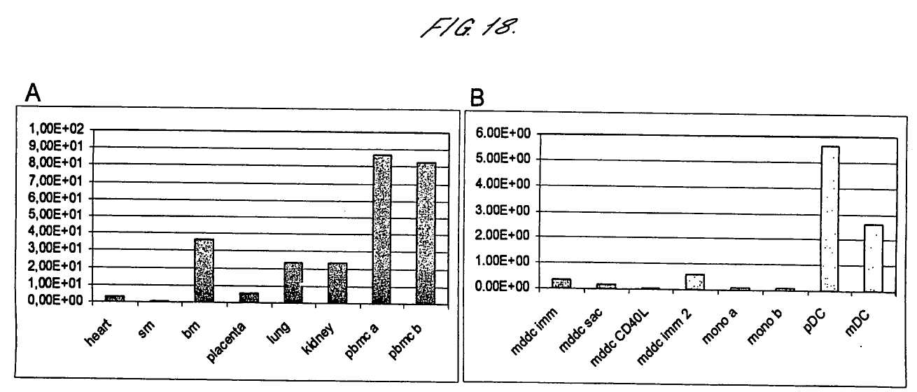

In order to determine where TREM-5 is most abundantly expressed, the inventors analyzed a pre-made MTE Array consisting of 75 tissue-specific poly A+ RNAs directly spotted on a nylon membrane. An extensive analysis of a wide variety of foetal and adult tissues, allowed showed that TREM-5 mRNA levels are relatively abundant in bone marrow and also detected, although at lower levels, in spleen, lymphnodes, peripheral blood leukocytes, lung and kidney (FIG. 18, panel A). Quantification of TREM-5 mRNA in different tissues is shown in FIG. 5. Analysis of different cell subsets within peripheral blood leukocytes has revealed that TREM-5 is expressed in monocyte-derived immature dendritic cells and down-regulated upon stimulation of dendiritic cells with maturation-inducing stimuli such as Staphilococcus aureus (SAC) and CD40 ligand (CD40L) (FIG. 18, panel B). TREM-5 is also expressed on resting dendritic cells directly isolated from peripheral blood, both on plasmacytoid dendritic cells (pDC) and myeloid dendritic cells (mDC).

The contents of all references, patents and published patent applications cited throughout this application are hereby incorporated by reference in their entireties.

Those skilled in the art will recognize, or be able to ascertain many equivalents to the specific embodiments of the invention described herein using no more than routine experimentation. Such equivalents are intended to be encompassed by the following claims.

1. An isolated nucleic acid molecule comprising a nucleotide sequence encoding a TREM-4 polypeptide having an amino acid sequence of SEQ ID NO:4 or 5.

2. The nucleic acid molecule of claim 1, wherein the molecule is DNA.

3. The nucleic acid molecule of claim 1, wherein the molecule is RNA.

4. The nucleic acid molecule of claim 1 or 2, wherein the molecule is genomic DNA.

5. The nucleic acid molecule of claim 1 having a nucleotide sequence of SEQ ID NO:1 or 2.

6. A vector containing the DNA of claim 2.

7. A host cell comprising the vector of claim 6.

8. A host cell comprising the nucleic acid molecule of claim 2 operably linked to a heterologous promoter.

9. The host cell of claim 8 which is a prokaryotic cell.

10. The host cell of claim 8 which is a eukaryotic cell.

11. The host cell of claim 10 which is a mammalian cell.

12. A method for producing a TREM-4 polypeptide comprising expressing the polypeptide encoded by the nucleic acid molecule from the host cell of claim 7 or 8 and recovering the polypeptide.

13. A method for preparing a cell or progeny thereof capable of expressing a polypeptide comprising transfecting the cell with the vector of claim 6.

14. An isolated nucleic acid molecule which hybridizes under moderately stringent conditions to the nucleic acid molecule of claim 1 or a complement thereof.

15. An isolated TREM-4 polypeptide having the amino acid sequence of SEQ ID NO:4 or 5.

16. An antibody which immunospecifically recognizes the polypeptide of claim 15, or an antigen-binding fragment of said antibody.

17. An antagonist of the polypeptide of claim 15.

18. An agonist of the polypeptide of claim 15.

19. A method for treating a subject having a disease or disorder associated with an aberrant level of TREM-4, said method comprising administering to the subject a therapeutically effective amount of a modulator of TREM-4 expression or activity.

20. The method of claim 19, wherein said modulator is a polypeptide having an amino acid sequence of SEQ ID NO:4 or 5.

21. The method of claim 19, wherein said modulator is an anti-TREM-4 antibody or antigen binding fragment thereof.

22. The method of claims 19 to 21, wherein said disease or disorder is an inflammatory disease or disorder.

23. The method of claim 19, wherein said disease or disorder is cancer.

24. The method of claim 19, wherein said disease or disorder is infertility.

25. The method of claim 19, wherein said disease or disorder is a germ cell tumor.

26. The method of claim 19, wherein said disease or disorder is a heart disease or disorder.

27. The method of claim 19, wherein said disease or disorder effects microvascular compartments.

28. The method of claim 20, wherein the therapeutically effective amount of the polypeptide is administered by providing the subject with DNA encoding the polypeptide and expressing the polypeptide in vivo.

29. A method for detecting the presence of the nucleic acid molecule of claim 1 in a sample, comprising:
(a) contacting the sample with a nucleic acid probe or primer which selectively hybridizes to said nucleic acid molecule; and
(b) determining whether the nucleic acid probe or primer binds to the nucleic acid molecule in the sample.

30. A method for detecting the presence of TREM-4 polypeptide of claim 15 in a sample, comprising:
(a) contacting the sample with a compound which selectively binds to the TREM-4 polypeptide; and
(b) determining whether the compound binds to the TREM-4 polypeptide in the sample.

31. The method of claim 30, wherein the compound which binds to the polypeptide is an antibody.

32. A method of contraception comprising administering to a male subject an amount of a modulator of TREM-4 sufficient to reduce spermatogenesis in said male subject.

33. The method of claim 32, wherein the modulator is a polypeptide having an amino acid sequence of SEQ ID NO:4 or 5.

34. The method of claim 32, wherein the modulator is an antibody of the polypeptide having an amino acid sequence of SEQ ID NO:4 or 5.

35. A pharmaceutical composition comprising the polypeptide of claim 15 and a pharmaceutically acceptable carrier.

36. A kit comprising a container containing a polypeptide of claim 15 and instructions for use.

37. A kit comprising a container containing a compound which selectively binds to a polypeptide of claim 15 and instructions for use.

38. The kit of claim 27, wherein the compound is an antibody.

39. An isolated nucleic acid molecule encoding a TREM-5 polypeptide having an amino acid sequence of SEQ ID NO:6.

40. The nucleic acid molecule of claim 39, wherein the molecule is DNA.

41. The nucleic acid molecule of claim 39, wherein the molecule is RNA.

42. The nucleic acid molecule of claim 39, wherein the molecule is genomic DNA.

43. The nucleic acid molecule of claim 39 having a nucleotide sequence of SEQ ID NO:3.

44. A vector containing the DNA of claim 39.

45. A host cell comprising the vector of claim 44.

46. A host cell comprising the nucleic acid molecule of claim 40 operably linked to a heterologous promoter.

47. The host cell of claim 46 is a prokaryotic cell.

48. The host cell of claim 46 is an eukaryotic cell.

49. The host cell of claim 48 is a mammalian cell.

50. A method for producing a TREM-5 polypeptide comprising expressing the polypeptide encoded by the DNA from the host cell of claim 45 or 46, and recovering the polypeptide.

51. A method for preparing a cell or progeny thereof capable of expressing a polypeptide comprising transfecting the cell with the vector of claim 44.

52. An isolated nucleic acid molecule which hybridizes under moderately stringent conditions to the nucleic acid molecule of claim 39 or a complement thereof.

53. An isolated TREM-5 polypeptide having the amino acid sequence of SEQ ID NO:6.

54. An antibody which immunospecifically recognizes the polypeptide of claim 53, or an antigen-binding fragment of said antibody.

55. An antagonist of the polypeptide of claim 53.

56. An agonist of the polypeptide of claim 53.

57. A method for treating a subject having a disease or disorder associated with an aberrant level of TREM-5, said method comprising administering to the subject a therapeutically effective amount of a modulator of TREM-5.

58. The method of claim 57, wherein said modulator is the polypeptide of claim 53.

59. The method of claim 57, wherein said modulator is a polypeptide having an amino acid sequence of SEQ ID NO:6.

60. The method of claim 57, wherein said modulator is an anti-TREM-5 antibody or antigen binding fragment thereof.

61. The method of claim 57, wherein said disease or disorder is an inflammatory disease or disorder or a disease or disorder associated with aberrant dendritic cell function.

62. The method of claim 57, wherein said disease or disorder is a skin disease selected from the group consisting of atopic dermatitis, familial psoriasis, and epidermodysplasia verruciformis.

63. A method of claim 58, wherein the therapeutically effective amount of the polypeptide is administered by providing the subject with DNA encoding the polypeptide and expressing the polypeptide in vivo.

64. A method for detecting the presence of the nucleic acid of claim 39 in a sample, comprising:
(a) contacting the sample with a nucleic acid probe or primer which selectively hybridizes to said nucleic acid; and
(b) determining whether the nucleic acid probe or primer binds to the nucleic acid in the sample.

65. A method for detecting the presence of the TREM-5 polypeptide of claim 53 in a sample, comprising:
(a) contacting the sample with a compound which selectively binds to said TREM-5 polypeptide; and
b) determining whether the compound binds to the TREM-5 polypeptide in the sample.

66. A pharmaceutical composition comprising the polypeptide of claim 53 and a pharmaceutically acceptable carrier.

67. A kit comprising a container containing a polypeptide of claim 53 and instructions for use.

68. A kit comprising a container containing a compound which selectively binds to a polypeptide of claim 53 and instructions for use.

69. The kit of claim 68, wherein the compound is an antibody.

70. Use of a modulator of TREM-4 or TREM-5 activity in medicine.

71. Use of a modulator of TREM-4 activity in the prevention or treatment of inflammatory disease, cancer, infertility, a germ cell tumor, a heart disease or disorder or a disease or disorder that effects microvascular compartments.

72. Use of a modulator of TREM-4 activity in the manufacture of a medicament for the prevention or treatment of inflammatory disease, cancer, infertility, a germ cell tumor, heart disease or disorder or a disease or disorder that effects microvascular compartments.

73. Use of a modulator of TREM-5 activity in the prevention or treatment of inflammatory disease or disorder, a disease or disorder associated with aberrant dendritic cell function or a skin disease selected from the group consisting of atopic dermatitis, familial psoriasis, and epidermodysplasia verruciformis.

74. Use of a modulator of TREM-5 activity in the manufacture of a medicament for the prevention or treatment of inflammatory disease or disorder, a disease or disorder associated with aberrant dendritic cell function or a skin disease selected from the group consisting of atopic dermatitis, familial psoriasis, and epidermodysplasia verruciformis.


SEQUENCE LISTING (58 sequences)

SEQ ID NO: 1 - DNA, 993 bp, Homo sapiens
gtgtgactgaagaggagctcacagttcccagcgtctgctcccacggtgtccagcgcccagaatgcggcttctggtcctgctatggggttggctgctgctcccaggttatgaagccctggaggggccagaggaaatcagcgggttcgaaggggacactgtgtccctgcagtgcacctacagggaagagctgaggggacaccggaagtactggtgcaggaagggtgggatcctcttctctcgctgctctggccacatctatgcagaagaagaaggccaggagacaatgaagggcaggttgtccatccgtgacagccgccaggagctctcgctcattgtgaccctgtggaacctcaccctgcaagacgctggggagtactggtgtggggtcgagaaacgggcccccgatgagtctttactgatctctctgttcgtctttccagggagctcccgcccccccatgcagctggactccacctcagcagaggacaccagtccagctctcagcagtggcagctctaagcccagggtgtccatcccgatggtccgcatactggccccagtcctggtgctgctgagccttctgtcagccgcaggcctgatcgccttctgcagccacctgctcctgtggagaaaggaagctcaacaggccacggagacacagaggaacgagaagttctgcctctcacgcttgaatcacagaacatcagagctggatccagatgaagcctttgagatctctagcctgacttttctctcagtttgaagcaaatcgcctttcagaacgcccaggggtcagggagctgttagagatgcactctccagggccccagacccagctgaaccctccaggggtggggtccagggactctggcagtgctacaaactctagtgtgcttttacaaaagatgagactgaggcccagagagggtaaaggtcttgcccatggtcacacagcaggtggtggcccaaacagagaacaggacccagaaaaag

SEQ ID NO: 2 - DNA, 942 bp, Homo sapiens
gtgtgactgaagaggagctcacagttcccagcgtctgctcccacggtgtccagcgcccagaatgcggcttctggtcctgctatggggttggctgctgctcccaggttatgaagccctggaggggccagaggaaatcagcgggttcgaaggggacactgtgtccctgcagtgcacctacagggaagagctgaggggacaccggaagtactggtgcaggaagggtgggatcctcttctctcgctgctctggccacatctatgcagaagaagaaggccaggagacaatgaagggcaggttgtccatccgtgacagccgccaggagctctcgctcattgtgaccctgtggaacctcaccctgcaagacgctggggagtactggtgtggggtcgagaaacgggcccccgatgagtctttactgatctctctgttcgtctttccagggagctcccgcccccccatgcagctggactccacctcagcagaggacaccagtccagctctcagcagtggcagctctaagcccagggtgtccatcccgatggtccgcatactggccccagtcctggtgctgctgagccttctgtcagccgcaggcctgatcgccttctgcagccacctgctcctgtggagaaaggaagctcaacaggccacggagacacagaggaacgagaagttctgcctctcacgcttgaactccctgatgttttctctgagcctgccttggctctgagaccaaggtggtgtggctgctctgggacccaggctgagggtggtgctctggggaccaggggcaagagaataagggtccctgtgcccattgtctggagctgggaacctgccccaaggctactgctgcaagaagagactgtgccctccgaggccagcactccaacggagggggctgcagcatgccacttcaggcaacagaagcccctgctggcc

SEQ ID NO: 3 - DNA, 651 bp, Homo sapiens
acgaggagccgggaaggcagagccatgtggctgccccctgctctgctccttctcagcctctcaggctgtttctccatccaaggcccagagtctgtgagagccccagagcaggggtccctgacggttcaatgccactataagcaaggatgggagacctacattaagtggtggtgccgaggggtgcgctgggatacatgcaagatcctcattgaaaccagagggtcggagcaaggagagaagagtgaccgtgtgtccatcaaggacaatcagaaagaccgcacgttcactgtgaccatggaggggctcaggcgagatgacgcagatgtttactggtgtgggattgaaagaagaggacctgaccttgggactcaagtgaaagtgatcgttgacccagagggagcggcttccacaacagcaagctcacctaccaacagcaatatggcagtgttcatcggctcccacaagaggaaccactacatgctcctggtatttgtgaaggtgcccatcttgctcatcttggtcactgccatcctctggttgaaggggtctcagagggtccctgaggagccaggggaacagcctatctacatgaacttctccgaacctctgactaaagacatggccacttagagagatggatctgcagagcct

SEQ ID NO: 4 - protein, 245 aa, Homo sapiens
MRLLVLLWGWLLLPGYEALEGPEEISGFEGDTVSLQCTYSLQCTYSLQCTYREELRGHRKYWCRKGGILFSRCSGHIYAEEEGQETMKGRLSIRDSRQELSLIVTLWNLTLQDAGEYWCGVEKRAPDESLLISLFVFPGSSRPPMQLDSTSAEDTSPALSSGSSKPRVSIPMVRILAPVLVLLSLLSAAGLIAFCSHLLLWRKEAQQATETQRNEKFCLSRLNHRTSELDPDEAFEISSLTFLSV

SEQ ID NO: 5 - protein, 222 aa, Homo sapiens [sequence omitted]

SEQ ID NO: 6 - protein, 259 aa, Homo sapiens [sequence omitted]

SEQ ID NO: 7 - protein, 115 aa, Homo sapiens
EALEGPEEISGFEGDTVSLQCTYREELRGHRKYWCRKGGILFSRCSGHIYAEEEGQETMKGRLSIRDSRQELSLIVTLWNLTLQDAGEYWCGVEKRAPDESLLISLFVFPGSSRP

SEQ ID NO: 8 - protein, 114 aa, Homo sapiens
FSIQGPESVRAPEQGSLTVQCHYKQGWETYIKWWCRGVRWDTCKILIETRGSEQGEKSDRVSIKDNQKDRTFTVTMEGLRRDDADVYWCGIERRGPDLGTQVKVIVDPEGAAST

SEQ ID NO: 9 - protein, 117 aa, Homo sapiens
FPLSHPMTVAGPVGGSLSVQCRYEKEHRTLNKFWCRPPQILRCDKIVETKGSAGKRNGRVSIRDSPANLSFTVTLENLTEEDAGTYWCGVDTPWLRDFHDPIVEVEVSVFPAGTTTA

SEQ ID NO: 10 - protein, 117 aa, Homo sapiens
SPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLND

SEQ ID NO: 11 - protein, 118 aa, Homo sapiens
QAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQ

SEQ ID NO: 12 - protein, 123 aa, Homo sapiens
SGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDA

SEQ ID NO: 13 - protein, 120 aa, Homo sapiens
ATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGT

SEQ ID NO: 14 - protein, 117 aa, Mus musculus
QAGDEEEHKCFLEGENLTLTCPYNIMLYSLSLKAWQRVRSHGSPETLVLTNTRKADFNVARAGKYLLEDYPTESVVKVTVTGLQRQDVGLYQCVVYLSPDNVIILRQRIRLAWCQGK

SEQ ID NO: 15 - protein, 16 aa, Homo sapiens
MRLLVLLWGWLLLPGY

SEQ ID NO: 16 - protein, 207 aa, Homo sapiens
EALEGPEEISGFEGDTVSLQCTYSLQCTYSLQCTYREELRGHRKYWCRKGGILFSRCSGHIYAEEEGQETMKGRLSIRDSRQELSLIVTLWNLTLQDAGEYWCGVEKRAPDESLLISLFVFPGSSRPPMQLDSTSAEDTSPALSSGSSKPRVSIPMVRILAPVLVLLSLLSAAGLIAFCSHLLLWRKEAQQATETQRNEKFCLSRLN

SEQ ID NO: 17 - protein, 4 aa, Homo sapiens
NLTL

SEQ ID NO: 18 - protein, 7 aa, Homo sapiens
  MISC_FEATURE (2)..(2): Variable consensus residue
DXGXYXC

SEQ ID NO: 19 - protein, 27 aa, Homo sapiens
SKPRVSIPMVRILAPVLVLLSLLSAAG

SEQ ID NO: 20 - protein, 50 aa, Homo sapiens
SLLSAAGLIAFCSHLLLWRKEAQQATETQRNEKFCLSRLNHRTSELDPDE

SEQ ID NO: 21 - protein, 16 aa, Homo sapiens
MRLLVLLWGWLLLPGY

SEQ ID NO: 22 - protein, 206 aa, Homo sapiens
EALEGPEEISGFEGDTVSLQCTYREELRGHRKYWCRKGGILFSRCSGHIYAEEEGQETMKGRLSIRDSRQELSLIVTLWNLTLQDAGEYWCGVEKRAPDESLLISLFVFPGSSRPPMQLDSTSAEDTSPALSSGSSKPRVSIPMVRILAPVLVLLSLLSAAGLIAFCSHLLLWRKEAQQATETQRNEKFCLSRLNSLMFSLSLPWL

SEQ ID NO: 23 - protein, 4 aa, Homo sapiens
NLTL

SEQ ID NO: 24 - protein, 7 aa, Homo sapiens
  MISC_FEATURE (2)..(2): Variable consensus residue
DXGXYXC

SEQ ID NO: 25 - protein, 27 aa, Homo sapiens
LAPVLVLLSLLSAAGLIAFCSHLLLWR

SEQ ID NO: 26 - protein, 39 aa, Homo sapiens
SHLLLWRKEAQQATETQRNEKFCLSRLNSLMFSLSLPWL

SEQ ID NO: 27 - protein, 21 aa, Homo sapiens
MWLPPALLLLSLSGCFSIQGP

SEQ ID NO: 28 - protein, 180 aa, Homo sapiens
ESVRAPEQGSLTVQCHYKQGWETYIKWWCRGVRWDTCKILIETRGSEQGEKSDRVSIKDNQKDRTFTVTMEGLRRDDADVYGEYWCGVEKRAPDESLLISLFVFPGSSRPPMQLDSTSAEDTSPALSSGSSKPRVSIPMWCGIERRGPDLGTQVKVIVDPEGAASTTASSPTNSNMAVFI

SEQ ID NO: 29 - protein, 6 aa, Homo sapiens
  MISC_FEATURE (1)..(1): Variable consensus residue
XGXYXC

SEQ ID NO: 30 - protein, 33 aa, Homo sapiens
SSKPRVSIPMWCGIERRGPDLGTQVKVIVDPEG

SEQ ID NO: 31 - protein, 29 aa, Homo sapiens
TQVKVIVDPEGAASTTASSPTNSNMAVFI

SEQ ID NO: 32 - DNA, 21 bp, Artificial
  Primer
aatgcggcttctggtcctgct

SEQ ID NO: 33 - DNA, 21 bp, Artificial
  Primer
atcagtaaagactcatcgggg

SEQ ID NO: 34 - DNA, 34 bp, Artificial
  oligo(dT)-adapter
actcgagtcgacatcgattttttttttttttttt

SEQ ID NO: 35 - DNA, 20 bp, Artificial
  Primer
agacgctggggagtactggt

SEQ ID NO: 36 - DNA, 17 bp, Artificial
  Primer
gtgggtcgagaaacggg

SEQ ID NO: 37 - DNA, 17 bp, Artificial
  Primer
gactcgagtcgacatcg

SEQ ID NO: 38 - DNA, 20 bp, Artificial
  Primer
cccagggtgtccatcccgat

SEQ ID NO: 39 - DNA, 20 bp, Artificial
  Primer
ccttgctccacaggagcagg

SEQ ID NO: 40 - DNA, 41 bp, Artificial
  Primer
tagtaggaattccccagaatgcggcttctggtcctgctatg

SEQ ID NO: 41 - DNA, 41 bp, Artificial
  Primer
tagtagaagcttatacttacccctgggcttagagctgccac

SEQ ID NO: 42 - DNA, 35 bp, Artificial
  Primer
tagtagagatcttatgaagccctggaggggccaga

SEQ ID NO: 43 - DNA, 33 bp, Artificial
  Primer
tagtaggtcgacggacaccctgggcttagagct

SEQ ID NO: 44 - DNA, 34 bp, Artificial; identical to SEQ ID NO: 34
  oligo(dT)-adapter

SEQ ID NO: 45 - DNA, 21 bp, Artificial
  Primer
acgaggagccgggaaggcaga

SEQ ID NO: 46 - DNA, 20 bp, Artificial
  Primer
aggctctgcagatccatctc

SEQ ID NO: 47 - DNA, 35 bp, Artificial
  Primer
tagatggaattcatgtggctgccccctgctctgct

SEQ ID NO: 48 - DNA, 43 bp, Artificial
  Primer
tagtagaagcttatacttaccgtagtggttcctcttgtgggag

SEQ ID NO: 49 - DNA, 39 bp, Artificial
  Primer
tagtagaagcttgagtctgtgagagccccagagcagggg

SEQ ID NO: 50 - DNA, 36 bp, Artificial
  Primer
tagtagtctagactctgcagatccatctctctaagt

SEQ ID NO: 51 - DNA, 21 bp, Artificial
  Primer
tgctctggccacatctatgca

SEQ ID NO: 52 - DNA, 21 bp, Artificial
  Primer
tttctcgaccccacaccagta

SEQ ID NO: 53 - DNA, 29 bp, Artificial
  Primer
gacggggtcacccacactgtgcccatcta

SEQ ID NO: 54 - DNA, 30 bp, Artificial
  Primer
ctagaagcatttgcggtggacgatggaggg

SEQ ID NO: 55 - DNA, 20 bp, Artificial
  Primer
cccctgctctgctccttctc

SEQ ID NO: 56 - DNA, 19 bp, Artificial
  Primer
cacccctcggcaccaccac

SEQ ID NO: 57 - DNA, 29 bp, Artificial
  Primer
gacggggtcacccacactgtgcccatcta

SEQ ID NO: 58 - DNA, 30 bp, Artificial; identical to SEQ ID NO: 54
  Primer